(19) United States
(12) Patent Application Publication
(10) Pub. No.: US 2026/0207570 A1
(43) Pub. Date: Jul. 23, 2026

(54) OPTIMIZED DOSING OF LERIGLITAZONE

(71) Applicant: MINORYX THERAPEUTICS S.L., Mataró Barcelona (ES)

(72) Inventors: Maria Pilar Pizcueta LALANZA, Barcelona (ES); Laura Pilar Rodriguez PASCAU, Barcelona (ES); Andreas LINDAUER, Liergues (FR); Sonia Maria POLI, Geneva (CH)

(21) Appl. No.: 19/144,282

(22) PCT Filed: Dec. 27, 2023
(86) PCT No.: PCT/IB2023/063288

§ 371 Date: Jun. 27, 2025

(30) Foreign Application Priority Data
Dec. 28, 2022 (EP) 22383298.1

(51) Int. Cl. A61K 31/4439; A61P 25/28

(52) U.S. Cl. CPC A61K 31/4439; A61P 25/28

(57) ABSTRACT

The present disclosure provides methods of treating a disease or disorder in a patient comprising administering an optimized dose of leriglitazone, or a pharmaceutically acceptable salt thereof, e.g., leriglitazone HCl, to a patient in need thereof. Leriglitazone is a PPAR-γ agonist that is used to treat a variety of diseases and disorders including, but not limited to, liver diseases, e.g., nonalcoholic steatohepatitis, lung diseases, e.g., acute respiratory distress syndrome and acute lung injury, and central nervous system diseases, e.g., X-linked adrenoleukodystrophy, adrenomyeloneuropathy, cerebral adrenoleukodystrophy, and Friedreich's Ataxia.

### FIELD OF DISCLOSURE

The present disclosure provides methods of treating a disease or disorder in a patient comprising administering an optimized dose of leriglitazone, or a pharmaceutically acceptable salt thereof, e.g., leriglitazone HCl, to a patient in need thereof. Leriglitazone can be used to treat a variety of diseases and disorders including, but not limited to, liver diseases, e.g., nonalcoholic steatohepatitis (NASH), lung diseases and disorders, e.g., acute respiratory distress syndrome and acute lung injury, and central nervous system diseases, e.g., X-linked adrenoleukodystrophy (X-ALD), adrenomyeloneuropathy (AMN), cerebral adrenoleukodystrophy (cALD), and Friedreich's Ataxia.

### BACKGROUND

5-[[4-[2-[5-(1-hydroxyethyl)pyridin-2-yl]ethoxy]phenyl]methyl]-1,3-thiazolidine-2,4-dione (referred to herein as “leriglitazone” or Compound (1)) is a metabolite of pioglitazone see, e.g., Sohda et al., Chem. Pharm. Bull. 43(12):2168-2172 (1995); Maeshiba et al., Arzneim.-Forsch Drug Res. 47(I):29-35 (1997) having selective peroxisome proliferator-activated receptor gamma (PPAR-γ) agonist activity. WO2015/150476 A1 discloses leriglitazone for use in the treatment of central nervous system diseases. WO2018/100557 discloses leriglitazone, for the treatment of nonalcoholic fatty liver disease (“NAFLD”), nonalcoholic steatohepatitis (“NASH”), and other diseases and disorders. WO2019/234689 discloses an algorithm-based method to administer leriglitazone based on steady-state plasma levels of leriglitazone and related pharmacokinetic parameters in a patient. There exists a need in the art for methods of administering safe and efficacious doses of leriglitazone and 5-[[4-[2-[5-(1-hydroxyethyl)pyridin-2-yl]ethoxy]phenyl]methyl]-1,3-thiazolidine-2,4-dione hydrochloride (referred to herein as “leriglitazone HCl”), especially to pediatric patients.

### SUMMARY

Leriglitazone is orally bioavailable and displays linear pharmacokinetics in humans. But variations in bioavailability due to drug clearance and other factors, e.g., weight, age, sex, and BMI, that increase or decrease leriglitazone plasma concentration among individual patients make it difficult to administer a therapeutically effective amount of leriglitazone using conventional dosing techniques. Moreover, patients treated with leriglitazone may suffer from severe disease, e.g., X-ALD, AMN, cALD, Friedreich's Ataxia, lung inflammation, or NASH. It is thus imperative that the correct initial dose of leriglitazone or leriglitazone HCl be administered in order to maximize efficacy and minimize toxic side effects in this group of patients.

In one aspect, the present disclosure provides a method of treating a disease or disorder in a patient in need thereof, the method comprising administering Compound (1), or a pharmaceutically acceptable salt thereof, to the patient, wherein the initial dose of Compound (1), or a pharmaceutically acceptable salt thereof, in mg, is determined according to Formula I:

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} (target area under the concentration-time curve) is about 40 to about 240 μg·h/mL of Compound (1).

In another aspect, SF is 0.7, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.9.

In another aspect, Compound (1) is administered to the patient and SF is 0.74.

In another aspect, Compound (1) HCl is administered to the patient and SF is 0.81.

In another aspect, the disease or disorder is a CNS disease or disorder and the AUC_{target} is 170±20% μg·h/mL.

In another aspect, the disease or disorder is a CNS disease or disorder, the patient is a pediatric patient, i.e., the patient is ≤17 years old, and the AUC_{target} is 170±20% μg·h/mL.

In another aspect, the disease or disorder is a CNS disease or disorder, the patient is an adult patient, i.e., the patient is >17 years old, and the AUC_{target} is 170±20% μg·h/mL.

In another aspect, the disease or disorder is a lung disease or disorder and the AUC_{target} is 170±20% μg·h/mL.

In another aspect, the disease or disorder is a lung disease or disorder, the patient is a pediatric patient, and the AUC_{target} is 170±20% μg·h/mL.

In another aspect, the disease or disorder is a lung disease or disorder, the patient is an adult patient, and the AUC_{target} is 170±20% μg·h/mL.

In another aspect, the disease or disorder is a CNS disease or disorder and the AUC_{target} is 200±20% μg·h/mL.

In another aspect, the disease or disorder is a CNS disease or disorder, the patient is an adult patient, and the AUC_{target} is 200±20% μg·h/mL.

In another aspect, the disease or disorder is a lung disease or disorder and the AUC_{target} is 200±20% μg·h/mL.

In another aspect, the disease or disorder is a lung disease or disorder, the patient is an adult patient, and the AUC_{target} is 200±20% μg·h/mL.

In another aspect, the disease or disorder is a CNS disease or disorder and the AUC_{target} is 100 to 135 μg·h/mL.

In another aspect, the disease or disorder is a CNS disease or disorder, the patient is a pediatric patient, and the AUC_{target} is 100 to 135 μg·h/mL.

In another aspect, the disease or disorder is a CNS disease or disorder, the patient is an adult patient, and the AUC_{target} is 100 to 135 μg·h/mL.

In another aspect, the disease or disorder is a lung disease or disorder and the AUC_{target} is 100 to 135 μg·h/mL.

In another aspect, the disease or disorder is a lung disease or disorder, the patient is a pediatric patient, and the AUC_{target} is 100 to 135 μg·h/mL.

In another aspect, the disease or disorder is a lung disease or disorder, the patient is an adult patient, and the AUC_{target} is 100 to 135 μg·h/mL.

In another aspect, the disease or disorder is a liver disease or disorder, e.g., NASH or NAFLD, and the AUC_{target} is 50±20% μg·h/mL.

In another aspect, the present disclosure provides a method of treating a disease or disorder in a patient in need thereof, the method comprising determining the AUC in the patient, e.g., about 0.5 to about 12 hours, e.g., about 0.5 hours, about 1 hour, about 2 hours, about 3 hours, about 4 hours, about 5 hours, or about 6 hours, after a dose of Compound (1), or a pharmaceutically acceptable salt thereof, and
(i) administering a higher dose of Compound (1), or a pharmaceutically acceptable salt thereof, if the AUC is less than 149 μg·h/mL;
(ii) administering a lower dose of Compound (1), or a pharmaceutically acceptable salt thereof, if the AUC is more than 221 μg·h/mL; and
(iii) administering the same dose of Compound (1), or a pharmaceutically acceptable salt thereof, if the AUC is between 150 and 220 μg·h/mL.

In another aspect, the present disclosure provides methods of treating a disease or disorder in a patient in need thereof, the method comprising determining the AUC of Compound (1) in the patient, e.g., about 0.5 to about 12 hours, e.g., about 0.5 hours, about 1 hour, about 2 hours, about 3 hours, about 4 hours, about 5 hours, or about 6 hours, after administration of a dose of Compound (1), or a pharmaceutically acceptable salt thereof; and
administering a higher dose of Compound (1), or a pharmaceutically acceptable salt thereof, if the AUC of Compound (1) is less than 149 μg·h/mL;
(ii) administering a lower dose of Compound (1), or a pharmaceutically acceptable salt thereof, if the AUC of Compound (1) is more than 191 μg·h/mL; and
(iii) administering the same dose of Compound (1), or a pharmaceutically acceptable salt thereof, if the AUC of Compound (1) is between 150 and 190 μg·h/mL,
wherein the patient is a pediatric patient.

In another aspect, the present disclosure provides methods of treating a liver disease or disorder, e.g., NASH, in a patient in need thereof, the method comprising determining the AUC of leriglitazone in the patient, e.g., about 0.5 to about 12 hours, e.g., about 0.5 hours, about 1 hour, about 2 hours, about 3 hours, about 4 hours, about 5 hours, or about 6 hours, after administration of a dose of leriglitazone or leriglitazone HCl; and
administering a higher dose of leriglitazone or leriglitazone HCl if the AUC of Compound (1) is less than 49 μg·h/mL;
(ii) administering a lower dose of leriglitazone or leriglitazone HCl if the AUC of leriglitazone is more than 101 μg·h/mL; and
(iii) administering the same dose of leriglitazone or leriglitazone HCl if the AUC of leriglitazone is between 50 and 100 μg·h/mL.

In another aspect, the present disclosure provides a method of treating a disease or disorder in a patient in need thereof, the method comprising:
(i) administering 10 mL of an oral suspension comprising 15 mg of leriglitazone HCl per mL to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) administering 12 mL of an oral suspension comprising 15 mg of leriglitazone HCl per mL to the patient after (i).

In another aspect, the present disclosure provides a method of treating a disease or disorder in a patient in need thereof, the method comprising:
(i) administering 10 mL of an oral suspension comprising 13.66 mg of leriglitazone per mL to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) administering 12 mL of an oral suspension comprising 13.66 mg of leriglitazone per mL to the patient after (i).

In another aspect, Compound (1), or a pharmaceutically acceptable salt thereof, is leriglitazone. In another aspect, Compound (1), or a pharmaceutically acceptable salt thereof, is leriglitazone HCl.

In another aspect, the present disclosure provides a method of treating a central nervous system disease or disorder, a mitochondrial disease, a liver disease or disorder, e.g., nonalcoholic steatohepatitis (NASH), nonalcoholic fatty liver disease (NAFLD), a chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

### BRIEF DESCRIPTION OF THE DRAWINGS

FIG. 1 is a line graph showing the ontogeny profile for CYP2C8 in pediatric subjects.

FIG. 2 is a line graph showing the ontogeny profile for CYP3A4 in pediatric subjects.

FIG. 3 is a line graph showing the ontogeny profile for relative CYP3A4/CYP2C8 in pediatric subjects.

FIG. 4 is an illustration showing the workflow for the Leriglitazone PBPK Model.

FIG. 5 is a line graph showing the mean plasma total concentration-time profiles of leriglitazone of SAD part in fasted conditions in semi-logarithmic scale.

FIG. 6 is a line graph showing the mean plasma total concentration-time profiles of M3 (5-[[4-[2-[5-acetylpyridin-2-yl]ethoxy]phenyl]methyl]-1,3-thiazolidine-2,4-dione) of SAD part in fasted conditions in semi-logarithmic scale.

FIG. 7 is an illustration comparing the neuro-PK in rat and phase 1 human data.

FIG. 8 is a line graph showing the plasma total concentration-time profiles (Mean+/−SD) of leriglitazone of MAD part on day 1 to day 13 in semi-logarithmic scale

FIG. 9 is a line graph showing the plasma total concentration-time profiles (Mean+/−SD) of M3 of MAD part on day 1 to day 13 in semi-logarithmic scale.

FIG. 10 is two line graphs showing simulated (lines; 10×6 virtual individuals) and observed (individual data points; from Phase 1 SAD part) plasma total concentration-time profiles of leriglitazone following a single 90 mg dose (fasted and fed) of leriglitazone HCl. The circles are the observed individual data. The grey lines represent the outcomes of simulated individual trials and the solid black line is the mean data for the simulated population (n=60). The grey dashed lines represent the 95th and 5th percentile of the simulated data.

FIG. 11 is two line graphs showing simulated (lines; 10×6 virtual individuals) and observed (individual data points; from Phase 1 SAD part) plasma total concentration-time profiles of leriglitazone following a single 270 mg dose (fasted and fed) of leriglitazone HCl. The circles are the observed individual data. The grey lines represent the outcomes of simulated individual trials and the solid black line is the mean data for the simulated population (n=60). The grey dashed lines represent the 95th and 5th percentile of the simulated data.

FIG. 12 is two line graphs showing simulated (lines; 10×8 virtual individuals) and observed (data points; from study Phase 1 MAD part) plasma concentration-time profiles of leriglitazone following multiple daily oral doses of 135 mg and 270 mg fed of leriglitazone HCl. The circles are the individual observed data. The grey lines represent the outcomes of simulated individual trials and the solid black line is the mean data for the simulated population (n=80). The grey dashed lines represent the 95th and 5th percentiles of the simulated data.

FIG. 13 is a line graph showing the dose linearity in drug exposure (AUC) in 6-12 year age range receiving 0.5 to 5 mg/kg.

FIG. 14 is six line graphs showing the observed leriglitazone total concentrations (black spots) overlaid on PBPK simulations using Upreti ontogeny for age group>2-6 year. Grey line is the median of simulations. Grey area is the 95% prediction Interval around the simulated concentrations.

FIG. 15 is two line graphs showing the observed leriglitazone total concentrations (black spots) overlaid on PBPK simulations using Upreti ontogeny for age group>6-12 year. Grey line is the median of simulations. Grey area is the 95% prediction Interval around the simulated concentrations.

FIG. 16 is two line graphs showing the observed leriglitazone total concentrations (black spots) overlaid on PBPK simulations using Upreti ontogeny for age group>12 year. Grey line is the median of simulations. Grey area is the 95% prediction Interval around the simulated concentrations.

FIG. 17 is a forest plot showing a comparison of the observed AUC versus the PBPK model median for each pediatric age group.

FIG. 18 is a forest plot showing a comparison of the AUC-body weight correlation of observed versus the PBPK model for each pediatric age group.

FIG. 19 is a forest plot showing a comparison of the Clearance-body weight correlation of observed versus the PBPK model for each pediatric age group.

FIG. 20 is a forest plot showing covariate effects on AUC following administration of leriglitazone.

### DETAILED DESCRIPTION

### I. Administration of Compound (1), or a Pharmaceutically Acceptable Salt Thereof

The methods of the present disclosure comprise administering 5-[[4-[2-[5-(1-hydroxyethyl)pyridin-2-yl]ethoxy]phenyl]methyl]-1,3-thiazolidine-2,4-dione, or a pharmaceutically acceptable salt thereof, to a patient in need thereof. 5-[[4-[2-[5-(1-hydroxyethyl)pyridin-2-yl]ethoxy]phenyl]methyl]-1,3-thiazolidine-2,4-dione may also be referred to as 5-(4-(2-(5-(1-hydroxyethyl)pyridine-2-yl)ethoxy)benzyl)thiazolidine-2,4-dione, hydroxypioglitazone, hydroxy pioglitazone, or M-IV. See, e.g., Sohda et al., Chem. Pharm. Bull. 43(12):2168-2172 (1995) and Maeshiba et al., Arzneim.-Forsch Drug Res. 47(I):29-35 (1997). In some embodiments, the methods of the present disclosure comprise administering leriglitazone to a patient in need thereof. In some embodiments, the methods of the present disclosure comprise administering leriglitazone HCl to a patient in need thereof.

5-[[4-[2-[5-(1-hydroxyethyl)pyridin-2-yl]ethoxy]phenyl]methyl]-1,3-thiazolidine-2,4-dione has two chiral centers. One of them is the carbon atom in the 5-position of the thiazolidine-dione ring and the other asymmetric atom is at position 1 of the hydroxyethyl group as shown by the arrows:

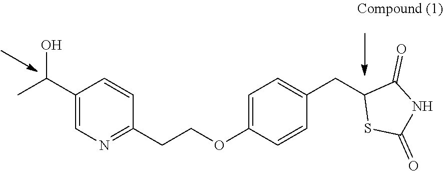 [SMILES: CC(O)c1ccc(CCOc2ccc(CC3SC(=O)NC3=O)cc2)nc1]

As used herein, the terms “5-[[4-[2-[5-(1-hydroxyethyl)pyridin-2-yl]ethoxy]phenyl]methyl]-1,3-thiazolidine-2,4-dione” or “Compound (1)” includes all possible stereoisomers, including enantiomers, see Compounds (2) to (5), below, and diastereomers, and mixtures thereof, including racemic and diastereomeric mixtures, of 5-[[4-[2-[5-(1-hydroxyethyl)pyridin-2-yl]ethoxy]phenyl]methyl]-1,3-thiazolidine-2,4-dione.

In one embodiment, the methods of the present disclosure comprise administering (R)-5-[[4-[2-[5-(R)-(1-hydroxyethyl)pyridin-2-yl]ethoxy]phenyl]methyl]-1,3-thiazolidine-2,4-dione (Compound (2)), or a pharmaceutically acceptable salt thereof, to a patient in need thereof.

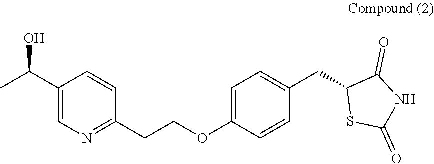 [SMILES: C[C@@H](O)c1ccc(CCOc2ccc(C[C@H]3SC(=O)NC3=O)cc2)nc1]

In another embodiment, the methods of the present disclosure comprise administering (R)-5-[[4-[2-[5-(S)-(1-hydroxyethyl)pyridin-2-yl]ethoxy]phenyl]methyl]-1,3-thiazolidine-2,4-dione (Compound (3)), or a pharmaceutically acceptable salt thereof, to a patient in need thereof.

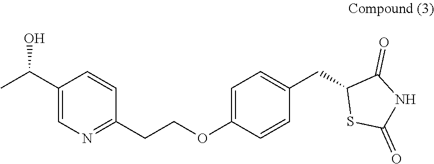 [SMILES: C[C@H](O)c1ccc(CCOc2ccc(C[C@H]3SC(=O)NC3=O)cc2)nc1]

In another embodiment, the methods of the present disclosure comprise administering (S)-5-[[4-[2-[5-(R)-(1-hydroxyethyl)pyridin-2-yl]ethoxy]phenyl]methyl]-1,3-thiazolidine-2,4-dione (Compound (4)), or a pharmaceutically acceptable salt thereof, to a patient in need thereof,

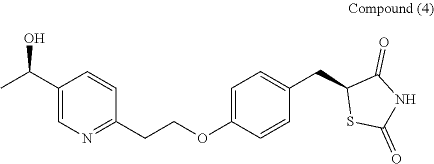 [SMILES: C[C@@H](O)c1ccc(CCOc2ccc(C[C@@H]3SC(=O)NC3=O)cc2)nc1]

In another embodiment, methods of the present disclosure comprise administering (S)-5-[[4-[2-[5-(S)-(1-hydroxyethyl)pyridin-2-yl]ethoxy]phenyl]methyl]-1,3-thiazolidine-2,4-dione (Compound (5)), or a pharmaceutically acceptable salt thereof, to a patient in need thereof.

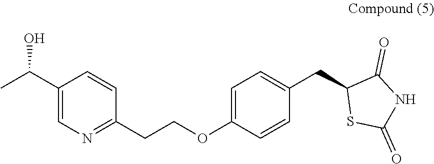 [SMILES: C[C@H](O)c1ccc(CCOc2ccc(C[C@@H]3SC(=O)NC3=O)cc2)nc1]

Compounds (2) to (5) have been prepared and isolated, see WO 2015/150476 A1, but their absolute (R/S) stereochemistry has not yet been determined. The retention time of each enantiomer has been measured by chiral HPLC.

Reference to Compounds (1) to (5) in the present disclosure is intended to designate these compounds as having hydrogen atoms which are predominantly in the form of its isotope ¹H, i.e. no more than 1% of the total number of hydrogen atoms per mole of compound are in the form of the ²H isotope (deuterium). In one embodiment, no more than 0.015% (which is the natural abundance of deuterium) of the total number of hydrogen atoms per mole of compound are in the form of the ²H isotope (deuterium).

In one embodiment, the patient is administered a mixture comprising a non-equimolar amount of each of Compound (2), or a pharmaceutically acceptable salt thereof, Compound (3), or a pharmaceutically acceptable salt thereof; Compound (4), or a pharmaceutically acceptable salt thereof, and Compound (5), or a pharmaceutically acceptable salt thereof.

In another embodiment, the patient is administered a mixture comprising each of Compound (2), or a pharmaceutically acceptable salt thereof; Compound (3), or a pharmaceutically acceptable salt thereof; Compound (4), or a pharmaceutically acceptable salt thereof; and Compound (5), or a pharmaceutically acceptable salt thereof, in an amount of 20%±10% w/w.

In another embodiment, the patient is administered a mixture comprising each of Compound (2), or a pharmaceutically acceptable salt thereof; Compound (3), or a pharmaceutically acceptable salt thereof; Compound (4), or a pharmaceutically acceptable salt thereof; and Compound (5), or a pharmaceutically acceptable salt thereof, in an amount of 25%±5% w/w.

In another embodiment, the patient is administered a mixture comprising each of Compound (2), or a pharmaceutically acceptable salt thereof; Compound (3), or a pharmaceutically acceptable salt thereof; Compound (4), or a pharmaceutically acceptable salt thereof, and Compound (5), or a pharmaceutically acceptable salt thereof, wherein the mixture comprises an enantiomeric excess of one or more of Compound (2), Compound (3), Compound (4), and Compound (5).

In another embodiment, the patient is administered a mixture comprising an equimolar amount of each Compound (2), or a pharmaceutically acceptable salt thereof; Compound (3), or a pharmaceutically acceptable salt thereof; Compound (4), or a pharmaceutically acceptable salt thereof, and Compound (5), or a pharmaceutically acceptable salt thereof, i.e., each compound, or pharmaceutically acceptable salt thereof, in an amount of 25% w/w.

In one embodiment, the patient is administered a mixture comprising on non-equimolar amount of Compound (2), or a pharmaceutically acceptable salt thereof, Compound (3), or a pharmaceutically acceptable salt thereof; Compound (4), or a pharmaceutically acceptable salt thereof, and Compound (5), or a pharmaceutically acceptable salt thereof, wherein the mixture is optically active.

In another embodiment, the mixture comprises:
(a) Compound (2), or a pharmaceutically acceptable salt thereof, and Compound (3), or a pharmaceutically acceptable salt thereof;
(b) Compound (4), or a pharmaceutically acceptable salt thereof, and Compound (5), or a pharmaceutically acceptable salt thereof;
(c) Compound (2), or a pharmaceutically acceptable salt thereof, and Compound (4), or a pharmaceutically acceptable salt thereof, and
(d) Compound (3), or a pharmaceutically acceptable salt thereof, and Compound (5),
wherein each compound, or a pharmaceutically acceptable salt thereof, is independently present in an equimolar or non-equimolar amount.

In another embodiment, the patient is administered the mixture (c) or the mixture (d) as these mixtures are defined above.

In another embodiment, the patient is administered a mixture consisting essentially of:
(a) Compound (2), or a pharmaceutically acceptable salt thereof, and Compound (3), or a pharmaceutically acceptable salt thereof, as the active agents;
(b) Compound (4), or a pharmaceutically acceptable salt thereof, and Compound (5), or a pharmaceutically acceptable salt thereof, as the active agents;
(c) Compound (2), or a pharmaceutically acceptable salt thereof, and Compound (4), or a pharmaceutically acceptable salt thereof, as the active agents; and
(d) Compound (3), or a pharmaceutically acceptable salt thereof, and Compound (5), or a pharmaceutically acceptable salt thereof, as the active agents.

In another embodiment of the mixtures (a) to (d) mentioned above, the two compounds mentioned in each one of the mixtures are present in equimolar quantities. Said mixtures may comprise also minor amounts (e.g., less than 10 wt. %, less than 3 wt. %, less than 1 wt. %, and less than 0.1 wt. % of another stereoisomer of formula (1)). Said mixtures can also be enantiomerically enriched with respect to one or more Compounds (2), (3), (4), and (5).

In another aspect of the disclosure, a pharmaceutically acceptable salt of Compound (1) is administered to the patient. Suitable pharmaceutically acceptable salts include, for example, pharmaceutically acceptable acid addition salts of Compound (1) prepared from the following acids: formic, acetic, propionic, benzoic, succinic, glycolic, gluconic, lactic, maleic, malic, tartaric, citric, nitric, ascorbic, glucuronic, maleic, fumaric, pyruvic, aspartic, glutamic, hydrochloric, hydrobromic, hydroiodic, isocitric, xinafoic, tartaric, trifluoroacetic, pamoic, anthranilic, mesylic, 1,5-naphthalenedisulfonic, oxalacetic, oleic, stearic, salicylic, p-hydroxybenzoic, nicotinic, phenylacetic, mandelic, embonic (pamoic), methanesulfonic, phosphoric, phosphonic, ethanesulfonic, benzenesulfonic, pantothenic, toluenesulfonic, 2-hydroxyethanesulfonic, sulfanilic, sulfuric, cyclohexylaminosulfonic, algenic, β-hydroxybutyric, galactaric and galacturonic acids. In an embodiment, the pharmaceutically acceptable acid addition salts include the salts of Compound (1) prepared from hydrochloric acid and hydrobromic acid. In one embodiment, the pharmaceutically acceptable salt of Compound (1) is the salt of the hydrochloric acid, e.g., 5-[[4-[2-[5-(1-hydroxyethyl)pyridin-2-yl]ethoxy]phenyl]methyl]-1,3-thiazolidine-2,4-dione hydrochloride.

Leriglitazone can be prepared by any suitable method known in the art, such as by the processes described in WO 2015/150476 A1 and WO 2018/116281 A1. Leriglitazone is also commercially available from, for example, Santa Cruz Biotechnology and Toronto Research Chemicals (Toronto, Ontario, Canada).

### II. Methods and Uses of the Disclosure

In one embodiment, the present disclosure provides methods of treating a disease or disorder in a patient in need thereof, the method comprising administering 5-[[4-[2-[5-(1-hydroxyethyl)pyridin-2-yl]ethoxy]phenyl]methyl]-1,3-thiazolidine-2,4-dione (Compound (1)), or a pharmaceutically acceptable salt thereof, to the patient, wherein the initial dose of Compound (1), or a pharmaceutically acceptable salt thereof, in mg, is determined according to Formula I:

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 240 μg·h/mL of Compound (1).

Formula I was derived from a population pharmacokinetic model, see Example 2, which was developed by jointly analyzing concentration-time data from three clinical studies in patients receiving Compound (1). In this model, the primary pharmacokinetic parameters clearance (CL) and bioavailability (F) were found to be significantly related to the covariates body weight, age, sex, BMI and prandial state. The general pharmacokinetic relationship between dose, CL, F and AUC can be expressed as: AUC=F×Dose/CL, and rearranged to: Dose=AUC×CL/F, in order to calculate the required dose to achieve a desired target AUC, given a patient individual CL and F. Formula I includes the calculation of a patient's CL (numerator) and F (denominator) as a function of his/her individual covariates as derived from the population pharmacokinetic model.

In another embodiment, the present disclosure provides Compound (1), or a pharmaceutically acceptable salt thereof, for use in treating a disease or disorder in a patient, wherein the initial dose of Compound (1), or a pharmaceutically acceptable salt thereof, in mg, is determined according to Formula I.

In another embodiment, BW_{coef} is about 0.6 to 0.9.

In another embodiment, AGE_{coef} is about 0.02 to 0.2.

In another embodiment, SEX_{coef} is about 0.04 to 0.25.

In another embodiment, BMI_{coef} is about 0.3 to 0.8.

In another embodiment, FOOD_{coef} is about 0.02 to 0.7.

In another embodiment, AUC_{target} is about 40 to 240 μg·h/mL of Compound (1).

In another embodiment, BW_{coef} is 0.6 to 0.9.

In another embodiment, AGE_{coef} is 0.02 to 0.2.

In another embodiment, SEX_{coef} is 0.04 to 0.25.

In another embodiment, BMI_{coef} is 0.3 to 0.8.

In another embodiment, FOOD_{coef} is 0.02 to 0.7.

In another embodiment, AUC_{target} is 40 to 240 μg·h/mL of Compound (1).

In another embodiment, the BW_{coef} is 0.75.

In another embodiment, the AGE_{coef} is 0.131.

In another embodiment, the SEX_{coef} is 0.147.

In another embodiment, the BMI_{coef} is 0.541.

In another embodiment, the FOOD_{coef}=0.0477.

In another embodiment, SF is 0.7, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.9.

In another embodiment, Compound (1), e.g., leriglitazone, is administered and SF is 0.74.

In another embodiment, Compound (1) HCl, e.g., leriglitazone HCl, is administered and SF is 0.81.

In another embodiment, the AUC_{target} is 50±20% μg·h/mL, 60±20% μg·h/mL, 70±20% μg·h/mL, 80±20% μg·h/mL, 90±20% μg·h/mL, 100±20% μg·h/mL, 110±20% μg·h/mL, 120±20% μg·h/mL, 130±20% μg·h/mL, 140±20% μg·h/mL, 150±20% μg·h/mL, 160±20% μg·h/mL, 170±20% μg·h/mL, 180±20% μg·h/mL, 190±20% μg·h/mL, or 200±20% μg·h/mL, and, optionally, depends on the disease or disorder to be treated and the age of the patient.

In another embodiment, the AUC_{target} is 50±10% μg·h/mL, 60±10% μg·h/mL, 70±10% μg·h/mL, 80±10% μg·h/mL, 90±10% μg·h/mL, 100±10% μg·h/mL, 110±10% μg·h/mL, 120±10% μg·h/mL, 130±10% μg·h/mL, 140±10% μg·h/mL, 150±10% μg·h/mL, 160±10% μg·h/mL, 170±10% μg·h/mL, 180±10% μg·h/mL, 190±10% μg·h/mL, or 200±10% μg·h/mL, and, optionally, depends on the disease or disorder to be treated and the age of the patient.

In another embodiment, the AUC_{target} is 40 μg·h/mL, 50 μg·h/mL, 60 μg·h/mL, 70 μg·h/mL, 80 μg·h/mL, 90 μg·h/mL, 100 μg·h/mL, 110 μg·h/mL, 120 μg·h/mL, 130 μg·h/mL, 140 μg·h/mL, 150 μg·h/mL, 160 μg·h/mL, 170 μg·h/mL, 180 μg·h/mL, 190 μg·h/mL, 200 μg·h/mL, 210 μg·h/mL, 220 μg·h/mL, 230 μg·h/mL, or 240 μg·h/mL, and, optionally, depends on the disease or disorder to be treated and the age of the patient.

In another embodiment, the disease or disorder is a CNS disease or disorder.

In another embodiment, the disease or disorder is a lung disease or disorder.

In another aspect, the disease or disorder is a liver disease or disorder.

In another embodiment, the patient is a pediatric patient.

In another embodiment, the patient is an adult patient.

In another embodiment, the disease or disorder is a CNS disease or disorder, the patient is a pediatric patient, and the AUC_{target} is 170±20% μg·h/mL.

In another aspect, the disease or disorder is a CNS disease or disorder, the patient is an adult patient, and the AUC_{target} is 200±20% μg·h/mL.

In another aspect, the disease or disorder is a lung disease or disorder, the patient is a pediatric patient, and the AUC_{target} is 170±20% μg·h/mL.

In another aspect, the disease or disorder is a lung disease or disorder, the patient is an adult patient, and the AUC_{target} is 200±20% μg·h/mL.

In another aspect, the disease or disorder is a CNS disease or disorder and the AUC_{target} is 100±20% μg·h/mL.

In another aspect, the disease or disorder is a lung disease or disorder and the AUC_{target} is 100±20% μg·h/mL.

In another aspect, the disease or disorder is a liver disease or disorder and the AUC_{target} is 50±20% μg·h/mL.

In another embodiment, the amount of Compound (1), or a pharmaceutically acceptable salt thereof, in mg, administered to the patient as the initial dose is administered to the patient in subsequent doses for up to one week, two weeks, three weeks, four weeks, five weeks, or six weeks, or more.

In another embodiment, the present disclosure provides methods of treating a disease or disorder in a patient in need thereof, the method comprising determining the AUC of Compound (1) in the patient, e.g., about 0.5 to about 12 hours, e.g., about 0.5 hours, about 1 hour, about 2 hours, about 3 hours, about 4 hours, about 5 hours, or about 6 hours, after administration of a dose of Compound (1), or a pharmaceutically acceptable salt thereof; and
administering a higher dose of Compound (1), or a pharmaceutically acceptable salt thereof, if the AUC of Compound (1) is less than 149 μg·h/mL;
(ii) administering a lower dose of Compound (1), or a pharmaceutically acceptable salt thereof, if the AUC of Compound (1) is more than 221 μg·h/mL; and
(iii) administering the same dose of Compound (1), or a pharmaceutically acceptable salt thereof, if the AUC of Compound (1) is between 150 and 220 μg·h/mL.

In another embodiment, the present disclosure provides methods of treating a disease or disorder in a patient in need thereof, the method comprising determining the AUC of Compound (1) in the patient, e.g., about 0.5 to about 12 hours, e.g., about 0.5 hours, about 1 hour, about 2 hours, about 3 hours, about 4 hours, about 5 hours, or about 6 hours, after administration of a dose of Compound (1), or a pharmaceutically acceptable salt thereof; and
(i) administering a higher dose of Compound (1), or a pharmaceutically acceptable salt thereof, if the AUC of Compound (1) is less than 149 μg·h/mL;
(ii) administering a lower dose of Compound (1), or a pharmaceutically acceptable salt thereof, if the AUC of Compound (1) is more than 191 μg·h/mL; and
(iii) administering the same dose of Compound (1), or a pharmaceutically acceptable salt thereof, if the AUC of Compound (1) is between 150 and 190 μg·h/mL,
wherein the patient is a pediatric patient.

In another embodiment, the present disclosure provides Compound (1), or a pharmaceutically acceptable salt thereof, for use in treating a disease or disorder in a patient, comprising determining the AUC of Compound (1) in the patient after a dose of Compound (1), or a pharmaceutically acceptable salt thereof, wherein:
(i) a higher dose of Compound (1), or a pharmaceutically acceptable salt thereof, is to be administered to the patient if the AUC of Compound (1) is less than 149 μg·h/mL;
(ii) a lower dose of Compound (1), or a pharmaceutically acceptable salt thereof, is to be administered to the patient if the AUC of Compound (1) is more than 221 μg·h/mL; and
(iii) the same dose of Compound (1), or a pharmaceutically acceptable salt thereof, is to be administered to the patient if the AUC of Compound (1) is between 150 and 220 μg·h/mL,
wherein the patient is a pediatric patient.

In another embodiment, the present disclosure provides Compound (1), or a pharmaceutically acceptable salt thereof, for use in treating a disease or disorder in a patient, comprising determining the AUC of Compound (1) in the patient after a dose of Compound (1), or a pharmaceutically acceptable salt thereof, wherein:
(i) a higher dose of Compound (1), or a pharmaceutically acceptable salt thereof, is to be administered to the patient if the AUC of Compound (1) is less than 149 μg·h/mL;
(ii) a lower dose of Compound (1), or a pharmaceutically acceptable salt thereof, is to be administered to the patient if the AUC of Compound (1) is more than 191 μg·h/mL; and
(iii) the same dose of Compound (1), or a pharmaceutically acceptable salt thereof, is to be administered to the patient if the AUC of Compound (1) is between 150 and 190 μg·h/mL, wherein the patient is a pediatric patient.

In another embodiment, the present disclosure provides methods treating a disease or disorder in a patient, e.g., a pediatric patient or an adult patient, in need thereof, the method comprising:
(i) administering 10 mL of an oral suspension comprising 15 mg of leriglitazone HCl per mL to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) administering 12 mL of an oral suspension comprising 15 mg of leriglitazone HCl per mL to the patient after (i).

In another embodiment, the present disclosure provides a method of treating a disease or disorder in a patient, e.g., a pediatric patient or an adult patient, in need thereof, the method comprising:
(i) administering 10 mL of an oral suspension comprising 13.66 mg of leriglitazone per mL to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) administering 12 mL of an oral suspension comprising 13.66 mg of leriglitazone per mL to the patient after (i).

In another embodiment, the present disclosure provides Compound (1), or a pharmaceutically acceptable salt thereof, for use in treating a disease or disorder in a patient, e.g., a pediatric patient or an adult patient, in need thereof, wherein:
(i) 10 mL of an oral suspension comprising about 15 mg of leriglitazone, or a pharmaceutically acceptable salt thereof, preferably leriglitazone HCl, per mL is to be administered to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) 12 mL of an oral suspension comprising about 15 mg of leriglitazone, or a pharmaceutically acceptable salt thereof, preferably leriglitazone HCl, per mL is to be administered to the patient to the patient after (i).

In another aspect, the present disclosure provides a method of treating a disease or disorder in a patient, e.g., a pediatric patient or an adult patient, in need thereof, the method comprising:
(i) administering 10 mL of an oral suspension comprising about 13.66 mg of leriglitazone per mL to the patient for 1 to 10 weeks, e.g., 1-4 weeks, e.g., 1-6 weeks, e.g., 1 week, 2 weeks, 3 weeks, 4 weeks, 5 weeks, 6 weeks, 7 weeks, 8 weeks, 9 weeks, or 10 weeks; and
(ii) administering 12 mL of an oral suspension comprising about 13.66 mg of leriglitazone per mL to the patient after (i).

In another embodiment, Compound (1), or a pharmaceutically acceptable salt thereof, is 5-[[4-[2-[5-(1-hydroxyethyl)pyridin-2-yl]ethoxy]phenyl]methyl]-1,3-thiazolidine-2,4-dione hydrochloride. 5-[[4-[2-[5-(1-hydroxyethyl)pyridin-2-yl]ethoxy]phenyl]methyl]-1,3-thiazolidine-2,4-dione hydrochloride is referred to herein as “leriglitazone HCl” or “MIN-102.”

In another embodiment, the leriglitazone HCl is administered to the patient as an oral suspension comprising about 15 mg of leriglitazone HCl per mL.

In another embodiment, the leriglitazone HCl is administered to the patient as a tablet, capsule, or other solid form comprising about 30 mg of leriglitazone HCl, about 60 mg of leriglitazone HCl, or about 90 mg of leriglitazone HCl.

### III. Diseases and Disorders

The methods and uses of the present disclosure comprise administering Compound (1), or a pharmaceutically acceptable salt thereof, e.g., leriglitazone or leriglitazone HCl, to a patient in need thereof, to treat a variety of diseases or disorders.

In one embodiment, the disease or disorder is regulated by peroxisome proliferator-activated receptor gamma (PPAR-γ). PPAR-γ regulates, inter alia, fatty acid storage and glucose metabolism, and has been implicated in the pathology of numerous diseases and disorders.

In another embodiment, the disease or disorder is a central nervous system disease or disorder, a mitochondrial disease, a liver disease, e.g., nonalcoholic steatohepatitis (NASH) and nonalcoholic fatty liver disease (NAFLD), chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, or a lung disease or disorder, e.g., an inflammatory respiratory disease, viral-induced inflammatory lung conditions or diseases, acute inflammation of the lung, or interstitial lung disease.

In another embodiment, the disease or disorder is a central nervous system disease or disorder.

In another embodiment, the disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

In another embodiment, the disease or disorder is a neurodegenerative disease.

In another embodiment, the disease or disorder is selected from the group consisting of Alzheimer's disease, Huntington's chorea, Parkinson's disease, multiple sclerosis, leukodystrophy, amyotrophic lateral sclerosis (ALS), degenerative ataxia, multiple system atrophy, and a motor neuron disease.

In another embodiment, the disease or disorder is selected from the group consisting of Alzheimer's disease, Huntington's chorea, Parkinson's disease, multiple sclerosis, neuromyelitis optica, leukodystrophy, ALS, degenerative ataxia, multiple system atrophy, NBIA (neurodegeneration and brain iron accumulation disorders), neuromyopathy, and a motor neuron disease.

In another embodiment, disease or disorder is metachromatic leukodystrophy.

In another embodiment, disease or disorder is cerebral adrenoleukodystrophy.

In another embodiment, disease or disorder is adrenomyeloneuropathy.

In another embodiment, the disease or disorder is X-linked adrenoleukodystrophy.

In another embodiment, the disease or disorder is degenerative ataxia. In another embodiment, the degenerative ataxia is Friedreich's ataxia.

In another embodiment, the disease or disorder is a motor neuron disease.

In another embodiment, the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

In another embodiment, the disease or disorder is a central nervous system disorder. In another embodiment, the central nervous system disorder is a cerebrovascular disease selected from the group consisting of global or local ischemia, intracerebral haemorrhage, stroke, and vascular dementia. In another embodiment, the central nervous system disorder is a viral disease selected from the group consisting of meningitis, encephalitis, rabies, measles, mumps, poliomyelitis, herpes simplex, and varicella zoster.

In another embodiment, the disease or disorder is a rare metabolic disease, e.g., a metabolic disease that affects less than 200,000 people in the United States, or a metabolic disease that affects not more than 5 in 10,000 people in the European Union. In another embodiment, the rare metabolic disease is selected from the group consisting of organic acidemias, fatty acid disorders and genetic mitochondrial disorders.

In another embodiment, the disease or disorder is a mitochondrial disease.

Mitochondria are tiny subunits present inside every cell of the human body except red blood cells. Mitochondria's main role is to transform food an oxygen that enter the cells into useful energy. Pyruvate uptake across the mitochondrial inner membrane is a central branch point in cellular energy metabolism with the ability to balance glycolysis and oxidative phosphorylation and poise catabolic an anabolic metabolism. (See, e.g., Divakaruni et al., PNAS 110(14):5422-5427 (2013)). The mitochondrial pyruvate carrier (MPC) is an inner-membrane transporter that facilitates pyruvate uptake from the cytoplasm to mitochondria. It is a central regulator of mitochondrial substrate utilization, and restrictions in mitochondrial pyruvate uptake can potentiate the use of fatty acids and a range of amino acids to fuel cellular energetics and biosynthesis. (See, e.g., Divakaruni et al., J. Cell Biol. (2017)).

The MPC contains two proteins, MPC1 and MPC2, that form a carrier complex in the inner mitochondrial membrane. MPC transports pyruvate into mitochondrial matrix that is required for pyruvate metabolism and is critical for metabolic pathways. (See, e.g., McCommis et al., Biochem. J. 466: 443-454 (2015)).

Mitochondrial diseases are a group of disorders, each of which involves a mitochondrial dysfunction. Mitochondrial diseases are chronic, genetic, and often inherited disorders that that occur when mitochondria fail to produce enough energy for the body to function properly. Mitochondrial diseases can be present at birth, but can also occur at any age. These diseases can affect the cells of the brain, nerves, muscles, kidneys, heart, liver, eyes, ears, and/or pancreas. Mitochondrial dysfunction occurs when the mitochondria do not work as well as they should due to another disease or condition. Mitochondrial disease refers to a heterogeneous group of disorders that include primary and secondary mitochondrial disorders (See e.g, Niyazov et al., Mol. Syndromol. 7:122-137 (2016)). Primary mitochondrial disorders can be due to germline mutations in mitochondrial DNA (mtDNA) and/or nuclear DNA (nDNA) genes either encoding OXPHOS (oxidative phosphorylation) proteins directly or they affect OXPHOS function by impacting production of the complex machinery needed to run the OXPHOS process. Secondary mitochondrial disorders by contrast occur in many pathologic processes not involving OXPHOS, including inherited diseases with germline mutations in non-OXPHOS genes. Secondary mitochondrial disorders can also be acquired secondary to adverse environmental effects which can cause oxidative stress. Many conditions can lead to secondary mitochondrial dysfunction including autism, Parkinson's disease, Alzheimer's disease, muscular dystrophy, Lou Gehrig's disease, diabetes and cancer.

In another embodiment, the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX deficient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

In another embodiment, the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

In another embodiment, the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

In another embodiment, the disease or disorder is a liver disease or disorder.

In another embodiment, the disease or disorder is nonalcoholic steatohepatitis (NASH).

In another embodiment, the disease or disorder is a lung disease or disorder.

In another embodiment, the disease or disorder is an inflammatory lung condition or disease caused by a viral infection is hyperinflammation.

In another embodiment, the inflammatory lung condition or disease caused by a viral infection is systemic inflammatory response syndrome (SIRS).

In another embodiment, the inflammatory lung condition or disease caused by a viral infection is acute respiratory distress syndrome (ARDS) or acute lung injury (ALI).

In another embodiment, the inflammatory lung condition or disease caused by a viral infection is pneumonia.

In another embodiment, the inflammatory lung condition or disease caused by a viral infection is a hyperinflammatory syndrome. In another embodiment, the hyperinflammatory syndrome is hypercytokinaemia or “cytokine storm.” In another embodiment, the hypercytokinaemia is associated with multiorgan failure. In another embodiment, the hyperinflammatory syndrome is haemophagocytic lymphohistiocytosis.

In another embodiment, the viral infection is caused by a double and single stranded DNA virus. Exemplary DNA viruses include, but are not limited to, chickenpox, human cytomegalovirus, herpes simplex virus type 1, adenovirus, papillovirus, varicell-zoster, cytomegalovirus, Epstein-Barr, smallpox, cow pox, vaccionia virus and parvovirus.

In another embodiment, the viral infection is caused by a RNA virus. Exemplary RNA viruses include, but are not limited to, coronavirus, respiratory syncytial virus, parainfluenza-3 virus, bovine viral diarrhea virus, Venezuelan equine encephalomyelitis virus, Dengue virus, yellow fever virus, Coxsackie B3 virus, encephalomyocarditis virus, influenza A virus, Zika virus, Ebola virus, Junin virus, Lassa Fever virus, Chikungunya virus reovirus, rotavirus, enterovirus, rhinovirus, hepatovirus, cardiovirus, aphthovirus, poliovirus, parechovirus, erbovirus, kobuvirus, teschovirus, coxsackie, Rubella virus, hepatitis C virus, Influenza virus A, influenzavirus B, influenzavirus C, isavirus, thogotovirus Measles virus, mumps virus, respiratory syncytial virus, Rinderpest virus, canine distemper virus, Rabies virus, Vesicular stomatitis, Marburg virus, hepatitis E virus, lentivirus (HIV), and hantavirus.

In another embodiment, the viral infection is caused by a reverse transcribing viruses such as HIV, caulimovirus, Cacao swollen-shoot virus (CSSV) and hepatitis B virus.

In another embodiment, the viral infection is caused by coronavirus.

In another embodiment, the coronavirus is an animal coronavirus.

In another embodiment, the coronavirus is a human coronavirus.

In another embodiment, the human coronavirus is HCoV-229E, HCoV-OC43, HCoV-NL63, HCoV-HKU1, SARS-CoV, MERS-CoV, or SARS-CoV-2, or a mutated strain of HCoV-229E, HCoV-OC43, HCoV-NL63, HCoV-HKU1, SARS-CoV, MERS-CoV, or SARS-CoV-2.

In another embodiment, the human coronavirus is HCoV-229E, HCoV-OC43, HCoV-NL63, HCoV-HKU1, SARS-CoV, MERS-CoV, or SARS-CoV-2.

In another embodiment, the human coronavirus is a mutated strain of HCoV-229E, HCoV-OC43, HCoV-NL63, HCoV-HKU1, SARS-CoV, MERS-CoV, or SARS-CoV-2.

In another embodiment, the human coronavirus is HCoV-229E.

In another embodiment, the human coronavirus is mutated strain of HCoV-229E.

In another embodiment, the human coronavirus is HCoV-OC43.

In another embodiment, the human coronavirus is mutated strain of HCoV-OC43.

In another embodiment, the human coronavirus is HCoV-NL63.

In another embodiment, the human coronavirus is mutated strain of HCoV-NL63.

In another embodiment, the human coronavirus is HCoV-HKU1.

In another embodiment, the human coronavirus is mutated strain of HCoV-HKU1.

In another embodiment, the human coronavirus is SARS-CoV.

In another embodiment, the human coronavirus is mutated strain of SARS-CoV.

In another embodiment, the human coronavirus is MERS-CoV.

In another embodiment, the human coronavirus is mutated strain of MERS-CoV.

In another embodiment, the human coronavirus is SARS-CoV-2.

In another embodiment, the human coronavirus is mutated strain of SARS-CoV-2.

In another embodiment, the disease or disorder is acute inflammation of the lung caused by a bacterial infection, a parasitic infection, any of the above-mentioned viral infections, Moldoveanu et al., J Inflamm Res 2:1-11 (2009), or any other cause. Rezoagli et al., Ann Transl Med 5(14):282 doi: 10.21037/atm.2017.06.62 (2017).

In another embodiment, the acute inflammation of the lung is pneumonia.

In another embodiment, the acute inflammation of the lung is ARDS. In another embodiment, the ARDS is not caused by a viral infection.

In another embodiment, the disease or disorder is an interstitial lung disease (ILD).

In another embodiment, the ILD is caused by drugs/chemicals, e.g., chemotherapy, environmental exposure; autoimmune disease; or any other, e.g., idiopathic, cause.

In another embodiment, the ILD is acute interstitial pneumonia, allergic bronchopulmonary aspergillosis, asbestosis, beryllium disease, autoimmune pulmonary alveolar proteinosis, Blau syndrome, bronchiolitis obliterans, bronchiolitis obliterans organizing pneumonia, chronic granulomatous disease, coal worker's pneumoconiosis, CREST syndrome, cryptogenic organizing pneumonia, cystic fibrosis, diffuse idiopathic pulmonary neuroendocrine cell hyperplasia, diffuse panbronchiolitis, fibrosing mediastinitis, Froster-Huch syndrome, idiopathic acute eosinophilic pneumonia, idiopathic pulmonary fibrosis (IPF), idiopathic pulmonary hemosiderosis, Kabuki syndrome, Kaolin pneumoconiosis, Kartagener syndrome, lung agenesis, Manouvrier syndrome, meconium aspiration syndrome, nontuberculous mycobacterial lung disease, pleuroparenchymal fibroelastosis, pulmonary alveolar microlithiasis, recurrent respiratory papillomatosis, respiratory distress syndrome, silicosis, tracheobronchomalacia, Wolf-Hirschhorn syndrome, or Young syndrome.

In another embodiment, the ILD is idiopathic pulmonary fibrosis (IPF).

### IV. Pharmaceutical Compositions and Use as a Medicament

Pharmaceutical compositions comprising Compound (1), or a pharmaceutically acceptable salt thereof, e.g., leriglitazone, and a pharmaceutically acceptable excipient, can be administered by any suitable route of administration. For example, any of oral, intraoral, topical, epicutaneous, subcutaneous, transdermal, intramuscular, parenteral, ocular, rectal, vaginal, inhalation, buccal, sublingual and intranasal delivery routes can be suitable.

The present disclosure also provides the use of Compound (1), or a pharmaceutically acceptable salt thereof, in the manufacture of a medicament for the treating a disease or disorder in a patient in need thereof.

In one embodiment, Compound (1), or a pharmaceutically acceptable salt thereof, is administered orally. Oral forms of pharmaceutical compositions can be solid or liquid. Suitable oral dosage forms include tablets, capsules, pills, granules, suspensions, emulsions, syrups or solutions. The pharmaceutical compositions may be a solid form selected from, e.g., tablets, capsules, pills, or granules. In an embodiment, the oral form is a tablet. In another embodiment, the oral form is an oral solution or suspension. These are advantageous when the patient has difficulty swallowing, for example as a result of the disease or for geriatric and pediatric use. Sublingual preparations are also advantageous.

The amount that is “effective” will vary from patient to patient, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. Thus, the dose of the active agent will depend on the nature and degree of the condition, the age and condition of the patient, and other factors known to those skilled in the art. A typical daily dosage is from 0.1 mg to 200 mg, such as from 20 mg to 200 mg, e.g., for an adult from 10 mg to100 mg given as a single dose with no further dosing or in multiple doses, for example one to three times per day. The compounds described herein may also be administered in daily doses of from 80 mg to 600 mg. In one embodiment, the daily dose for an adult is from 50 mg to 300 mg. In one embodiment, the daily dose for an adult is about 90 mg, about 120 mg, about 150 mg, about 180 mg, or about 210 mg. A daily dose for a child is from 0.1 mg to 200 mg. In another embodiment, the daily for a child is from 10 mg to 100 mg.

The pharmaceutical compositions may contain conventional excipients known in the art and may be prepared by conventional methods. A specific compound or mixture of compounds may be selected for a particular route of delivery. Some compounds or mixtures of compounds may also be suitable based on their use to treat NAFLD and NASH, X-ALD, AMN, cALD, or other diseases or disorders.

Oral dosage forms may be prepared by combining Compound (1), or a pharmaceutically acceptable salt thereof, in an intimate admixture with at least one excipient according to conventional pharmaceutical compounding techniques. Excipients can take a wide variety of forms depending on the form of the composition desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, kaolin, diluents, granulating agents, lubricants, binders, stabilizers, and disintegrating agents.

Due to their ease of administration, tablets, caplets (a coated oral tablet), and capsules (such as hard gelatin, HPMC, or starch capsules) represent an embodiment of the solid oral dosage unit forms, in which case solid pharmaceutical excipients are used. If desired, tablets or caplets can be coated by standard aqueous or nonaqueous techniques. These dosage forms can be prepared by any of the methods of pharmacy. In general, pharmaceutical compositions and dosage forms are prepared by uniformly and intimately admixing one or more compounds with liquid carriers, finely divided solid carriers, or both, and then shaping the product into the desired presentation if necessary.

For example, a tablet can be prepared by compression or molding. Compressed tablets can be prepared by compressing in a suitable machine one or more Compound (1), or a pharmaceutically acceptable salt thereof, in a free-flowing form, such as a powder or granules, optionally mixed with one or more excipients. Molded tablets can be made by molding in a suitable machine a mixture of the powdered compound moistened with an inert liquid diluent.

The pharmaceutical compositions may further comprise one or more other therapeutic agents. Combination treatments may be administered simultaneously, sequentially, or separately, by the same or by different routes, or before, during, and after surgical or intervention procedures.

In one embodiment, the present disclosure provides a pharmaceutical composition comprising leriglitazone HCl as an oral aqueous suspension.

In another embodiment, the present disclosure provides a pharmaceutical composition comprising leriglitazone HCl, Polysorbate 80, carboxymethylcellulose sodium and water.

In another embodiment, the present disclosure provides a pharmaceutical composition comprising leriglitazone HCl, colloidal microcrystalline cellulose, and carboxymethylcellulose sodium.

The pharmaceutical compositions of the present disclosure comprising Compound (1) HCl may also, optionally, comprise sweeting agents, e.g., sorbitol powder, saccharin sodium, preservatives, e.g., sodium benzoate, flavorings, pH regulators, e.g., sodium citrate, citric acid monohydrate.

Leriglitazone, or a pharmaceutically acceptable salt thereof, can be used according to the disclosure when the patient is also administered or in combination with one or more of another therapeutic agent selected from antiinflammatory and analgesic agents, antidiabetics (e.g., metformin), dopamine agonists (e.g. levodopa), MAO-B inhibitors, catechol O-methyltransferase (COMT) inhibitors, anticholinergics, other antiparkinsonians (e.g. amantadine), antiNMDA receptors (e.g. memantine), cholinesterase inhibitors, ACE inhibitors, glutamate antagonist (e.g. riluzole), antioxidants, immunomodulators (e.g. fingolimod, anti CD52, CD25 and CD20 monoclonal antibodies, interferon-β-1a, natalizumab, laquinimod, dimethylfumarate) chemotherapeutics, enzyme replacement therapy agents, substrate reduction therapy agents, corticosteroids, antiproliferatives (e.g. methotrexate), anticonvulsant medications, anticoagulants, antihypertensives, neuroprotectives, and NRf2 activators. Leriglitazone, or a pharmaceutically acceptable salt thereof, may also be used when the patient is undergoing gene therapy, bone marrow transplantation, deep brain stimulation or radiotherapy.

The one or more therapeutic agents include a sulfonylurea (e.g., glimepiride, glipizide, glyburide), a glinidine (also known as meglitinides), a thiazolidinedione (e.g., pioglitazone, rosiglitazone, lobeglitazone), a dipeptidyl peptidase 4 (DPP4) inhibitor (e.g., sitagliptin, vildagliptin, saxagliptin, linagliptin, gemigliptin, anagliptin, teneligliptin, alogliptin, trelagliptin, dutogliptin, omarigliptin), a sodium/glucose cotransporter 2 (SGLT2) inhibitor (e.g., canagliflozin, dapagliflozin), a glucagon-like peptide-1 (GLP1) receptor agonist (e.g., exenatide, liraglutide, lixisenatide, albiglutide, dulaglutide, taspoglutide, semaglutide), glucagon like peptide-1 (GLP-1), and insulin (e.g., animal insulin preparations extracted from the pancreas of cattle or pigs; human insulin preparations synthesized by genetic engineering using Escherichia coli or yeast; insulin zinc; protamine insulin zinc; insulin fragments or derivatives (e.g., INS-1), and oral insulin preparations.

In some embodiments, leriglitazone, or a pharmaceutically acceptable salt thereof, is administered to a patient having an inflammatory lung condition or disease caused by a viral infection, acute inflammation of the lung, or ILD in combination with one or more optional therapeutic agents. Optional therapeutic agents include hydroxychloroquine, choloroquine, antiviral agents such as Remdesivir and Favipiravir, IL6 inhibitors such as Kevzara and Actemra (Roche), corticoids, anticytokine inhibitors such as anakinra, JAK inhibitors, antibiotic agents, and antifungal agents.

Non-limiting exemplary antiviral agents include oseltamivir, ganciclovir, lopinavir/ritonavir (Kaletra®), and remdesivir. Antiviral agents also include reverse transcriptase inhibitors (RTIs). In one embodiment, the RTI is a nucleoside reverse transcriptase inhibitor (NRTI). Non-limiting exemplary NRTIs include abacavir (ZIAGEN™), abacavir/lamivudine (Epzicom), abacavir/lamivudine/zidovudine (TRIZIVIR™), adefovir, alovudine, amdoxovir, apricitabine, ATRIPLA®, BARACLUDE®, BIKTARVY®, censavudine, COVIRACIL™, DAPD/DXG, D-D4FC, dexelvucitabine, didanosine (VIDEX™), didanosine extended-release (Videx EC), dOTC, EFdA, emtricitabine (EMTRIVA™), emtricitabine/tenofovir alafenamide (DESCOVY®), emtricitabine/tenofovir disoproxil fumarate (TRUVADA®), elvucitabine, fosalvudine, lamivudine/zidovudine (COMBIVIR™), EVIPLERA™, GENVOYA®, HMD™ KIVEXA™, lamivudine (EPIVIR™), LODENOSINE™, ODEFSEY®, PREVEON®, racivir, stampidine, stavudine (ZERIT™), STRIBILD®, TENOFOVIR™, tenofovir disoproxil fumarate (VIREAD™), TRIUMEQ®, Trizivir, VEMLIDY®, and zidovudine (RETROVIR™). In another embodiment, the RTI is a non-nucleoside reverse transcriptase inhibitor (NNRTI). Non-limiting exemplary NNRTIs include delavirdine, efavirenz, etravirine, nevirapine, and rilvipirine. Antiviral agents also include protease inhibitors. Non-limiting exemplary protease inhibitors include amprenavir, fosamprenavir, indinavir, nelfinavir, saquinavir, atazanavir, darunavir, and tipranavir.

In one embodiment, the one or more optional therapeutic agents comprise Merimepodib, Tocilizumab (Actemra®), Favipiravir (Avigan®), Tocilizumab/favipiravir, Leronlimab (PRO 140), Remdesivir, Ruxolitinib (Kevzara®), Sarilumab, Chloroquine phosphate (Aralen®, Resochin®), Chloroquine hydrochloride, Azithromycin (Zithromax®), hydroxychloroquine sulfate/azithromycin, Lopinavir/Ritonavir (Kaletra®), Eculizumab (Soliris®), Human monoclonal antibody targeting SARS-CoV-2, APN01, Danoprevir (Ganovo®), TJM2 (TJ003234), Selinexor (XPOVIO®), Remestemcel-L (RYONCIL™), LAM-002 (apilimod), Rintatolimod (Ampligen®), DAS181, CM4620-IE, CAP-1002, SAB-185, ENU200, Camostat mesylate, IFX-1, Namilumab (IZN-101), GIAPREZA™ (angiotensin II), MN-166 (ibudilast), Rebif® (interferon beta-la), Ivermectin (Stromectol®, Mectizan®), NVX-CoV2373, Thiolanox®, Plitidepsin (Aplidin®), Opaganib (Yeliva®), RHB-107, Opaganib/RHB-107, EIDD-2801, Gimsilumab, TAK-888, ARMS-1, GENOSYL® (nitric oxide) gas, INOpulse®, BPI-002, rhu-pGSN, Galidesivir (BCX4430), BXT-10, L-glutamine oral powder (Endari®), Sylvant (siltuximab), Linebacker, Equivir, HTCC (N-(2-hydroxypropyl)-3-trimethylammonium 47 chitosan chloride), Darunavir (Prezista®), Darunavir/cobicistat (Prezcobix™), INOmax® (nitric oxide), WP1122, OYA1, Arbidol (umifenovir), Remescor®, MAN-01, STI-4920 (CMAB020), TZLS-501, IFN-alpha2b, Niclosamide, KL4, or WP1122.

Leriglitazone, or a pharmaceutically acceptable salt thereof, and the one or more optional therapeutic agents can be administered in combination under one or more of the following conditions: at different periodicities, at different durations, at different concentrations, by different administration routes, etc.

### V. Definitions

Various examples and embodiments of the inventive subject matter disclosed here are possible and will be apparent to a person of ordinary skill in the art, given the benefit of this disclosure. In this disclosure reference to “some embodiments,” “certain embodiments,” “certain exemplary embodiments,” “particular embodiments,” and similar phrases each means that those embodiments are non-limiting examples of the inventive subject matter, and there are alternative embodiments which are not excluded.

The articles “a,” “an,” and “the” are used herein to refer to one or to more than one (i.e., to at least one) of the grammatical object of the article. By way of example, “an element” means one element or more than one element.

The term “about,” as used herein, includes the recited number±10%. Thus, “about 10” means 9 to 11.

The word “comprising” is used in a manner consistent with its open-ended meaning, that is, to mean that a given product or process can optionally also have additional features or elements beyond those expressly described. It is understood that wherever embodiments are described with the language “comprising,” otherwise analogous embodiments described in terms of “consisting of” and/or “consisting essentially of” are also contemplated and within the scope of this disclosure.

The term “ameliorate” in the context of this present disclosure is understood as meaning any improvement on the situation of the patient treated.

The term “bid administration” or “BID” means twice daily administration of a therapeutic.

The term “SAD” means a single oral dose administration of a therapeutic.

By an “effective” amount or a “therapeutically effective amount” of a drug or pharmacologically active agent is meant a nontoxic but sufficient amount of the drug or agent to provide the desired effect. The amount that is “effective” will vary from patient to patient, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “treatment” or “to treat” and similar terms in the context of this specification means to ameliorate or eliminate the disease or one or more symptoms associated with said disease. “Treatment” also encompasses ameliorating or eliminating the physiological sequelae of the disease.

The term “pharmaceutically acceptable salt” refers to salts prepared from pharmaceutically acceptable inorganic and organic acids.

The term “prevention” or “to prevent” refers to the reduction in the risk of acquiring or developing a given disease or disorder, or the reduction or inhibition of the recurrence or a disease or disorder.

As used herein, the phrase “PK variability” or “pharmacokinetic variability” refer to inter-individual variations of a drugs pharmacokinetic parameters, resulting in different plasma concentration-time profiles after administration of the same dose to different patients.

As used herein, the term “steady-state” refers to the pharmacokinetic situation when the rate of drug administration is equal to the rate of drug elimination.

As used herein, the term “AUC” or refers to the area under the plasma (or blood) concentration time curve of leriglitazone, representing the exposure to the compound within the relevant time interval.

As used herein, the terms “AUC at steady-state” or “AUCₛₛ” refer to the AUC within a dosing interval at steady-state.

As used herein, the terms “trough value at steady state” or “C_{min ss}” refer to minimum steady-state plasma drug concentration during a dosage interval.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as |R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography or optical polarimetry.

The terms “enantiomerically pure” or “enantiopure” refer to a sample of a chiral substance all of whose molecules (within the limits of detection) have the same chirality sense.

The terms “enantiomerically enriched” or “enantioenriched” refer to a sample of a chiral substance whose enantiomeric ratio is greater than 50:50. Enantiomerically enriched compounds may be enantiomerically pure.

The term “primary mitochondrial disorder” or “PMD” refers to a mitochondrial disease that can occur due to germline mutations in mitochondrial DNA (mtDNA) and/or nuclear DNA (nDNA) genes encoding the electron transport chain (ETC) proteins and therefore the production of adenosine-triphosphate (ATP), the major cellular energy carrier.

The term “secondary mitochondrial disorder” or “SMD” refers to a mitochondrial disease accompanying many pathologic processes not involving oxidative phosphorylation (OXPHOS), including inherited diseases with germline mutations in non-OXPHOS genes. SMD can also be acquired secondary to adverse environmental effects which can cause oxidative stress.

The term “initial dose” refers to the first dose of the drug administered to a patient.

The term “subsequent dose” refers any dose of the drug administered to a patient after the initial dose.

The term “pediatric patient” as used herein refers to a patient that is ≤17 years old. In some embodiments, a pediatric patient is between 2 months and 17 years old. In some embodiments, a pediatric patient is between 6 months and 17 years old.

The term “adult patient” as used herein refers to a patient that is >17 years old.

### EXAMPLES

The methods of treatment or prevention and uses described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein. Formula I, see above, was created based Example 2.

### Example 1

### Physiologically-Based Pharmacokinetics (PBPK) Modelling to Guide Leriglitazone HCl Starting Dose in Pediatric Patients

### Phase 1 Study

Study design: A phase 1, randomized, double blind, placebo-controlled, single-center clinical study in 33 male healthy volunteers (HV) (19-54 years, 63.2-95.7 Kg) was conducted with leriglitazone HCl and divided in two parts: a single ascending dose (SAD) with three dose levels (30, 90 and 270 mg) in fasted conditions, followed with two dose levels (90 and 270 mg) in fed conditions; a multiple ascending dose (MAD) with a double-blind, parallel-design with two dose levels (135 and 270 mg) for 8 days and a separate cohort of subjects in an open-label parallel design with two dose levels (135 and 270 mg) including cerebrospinal fluid (CSF) collection on day 8. In the fasted conditions a fasting period of at least 4 hours was required before obtaining plasma samples. The food effect (FE) was assessed by comparing fasted with fed conditions (dosing after a high-fat breakfast per FDA definition with a calculated caloric content of 918 kcal). In the MAD part (fed) the medication was taken before a normal breakfast. Leriglitazone HCl was supplied by ChemConnection and an oral suspension of leriglitazone (20% w/v kleptose, 2% ethanol in 100 mM phosphate buffer pH=7.8) at 6 mg/ml was used in the study.

Safety: Safety and tolerability assessments consisted of adverse events (AEs), clinical laboratory, vital signs, 12-lead electrocardiogram (ECG), and physical examination.

Blood, Urine and CSF sampling: In the SAD, blood samples were drawn at the following time points: pre-dose and 0.25, 0.5, 0.75, 1, 1.5, 2, 2.5, 3, 4, 5, 6, 8, 10, 12, 16, 24, 36, 48, 72, 96 and 120 hours post-dose. Urine was collected at pre-dose (within 12 hours prior to dosing) and over 0-6, 6-12, 12-24, 24-36, 36-48 hours post-dose collection intervals. In the MAD, blood samples were collected on day 1 at pre-dose and 0.25, 0.5, 0.75, 1, 1.5, 2, 2.5, 3, 4, 5, 6, 8, 10, 12, and 16 hours post-dose and at pre-dose and at 4 hours post-dose on day 2 to day 7, and on day 8 at pre-dose and 0.25, 0.5, 0.75, 1, 1.5, 2, 2.5, 3, 4, 5, 6, 8, 10, 12, 16, 24, 36, 48, 72, 96 and 120 hours post-dose. CSF were taken on day −1 and on day 8, at 4 hours post-dose.

Pharmacokinetic parameter calculation: PK parameters (Cₘₐₓ, Tₘₐₓ, t_{1/2}, AUC₀₋ₜ, CL/F, Vz/F, AUC₀₋ₜ, R_{AUC}, RCₘₐₓ) were estimated using non-compartmental analysis (NCA) (Phoenix WinNonlin® Version 6.3).

Statistical analysis: Descriptive statistics for all relevant PK parameters: n, mean, minimum, median, maximum, geometric mean, and coefficient of variation (CV %); analysis of variance on Cₘₐₓ and AUC to determine dose proportionality and food effect (FE).

### Brain Distribution

The ratio of unbound leriglitazone concentration in brain to unbound concentration in plasma (Kpu,u; Cb,u/Cp,u) was calculated dividing the unbound CSF Cₘₐₓ by the unbound plasma Cₘₐₓ. Fractions unbound in plasma (fu,p) to obtain the unbound Cₘₐₓ values were already reported. Rodriguez-Pascau, L. et al., Sci. Transl. Med. 13, eabc0555 (2021).

### PBPK Model Development

A PBPK model for leriglitazone HCl was developed with the Simcyp Population-Based Simulator Version 17 release 1.

All input parameters for the PBPK model of leriglitazone are listed in Table 1.

TABLE 1

| Parameter | Leriglitazone |
|---|---|
| MW | 372.9 |
| log P | 2.47 |
| Compound type | Diprotic Base |
| pKa | 4.97 and 6.42 |
| B:P | 0.73 |
| fu | 0.04 |
| Vₛₛ (L/kg) | 0.29 |
| fa | 1 (CV: 10%) |
| T_{lag} (h) | 0.213 |
| ka (h1) | 0.49 to 6.35 |
| fu_{gut} | 1 |
| CL_{int, additional} (μl/min/mg) | 0.281 |
| CL_{int, CYP3A4} (μl/min/pmol) | 0.0097 |
| CL_{int, CYP2CB} (μl/min/mg) | 0.119 |
| CL_{int, bile} (μ/min/millions) | 0.358 |
| CL_{R} (L/h) | 0.033 |

A first-order absorption model with inputs based on clinical data was used to describe the absorption kinetics of the drug. Dose-dependent ka values were estimated from phase 1 SAD data and were incorporated to account for the less than dose proportional increase in the Cₘₐₓ of leriglitazone as well as the negative food effect on the Cₘₐₓ (Table 2). The use of a MechPeff model, which relies on physchem properties of the drug (log P), predicted a Fa value of 0.99, which was consistent with in vitro passive permeability data, and the fa value was set to be 1.0 in the final model.

TABLE 2

| Dose | Fasted/Fed | Vss (L) | Vss (L/kg) | Ka (1/h) |
|---|---|---|---|---|
| 30 mg | Fasted | 23.1 | 0.29 (9.1%) | 6.35 (CV: 676%) |
| 90 mg | Fasted | 25.2 | 0.31 (2.8%) | 4.43 (CV: 222%) |
| 270 mg | Fasted | 22.7 | 0.27 (12.1%) | 1.92 (CV: 75%) |
| Mean | | | 0.29 | |
| 90 mg | Fed | 28.1 | 0.35 (6.3%) | 0.89 (CV: 68%) |
| 270 mg | Fed | 24.4 | 0.30 (15.2%) | 0.49 (CV: 9%) |
| Mean | | | 0.32 | |

The estimated Vss using NCA (Phoenix WinNonlin Version 8.0) from the phase 1 SAD data was 0.29 L/kg (Table 2). In the PBPK model Method 3 was used because the predicted Vss (=0.39 L/kg) was the closest to the estimated Vss. A global scalar of 0.7 was applied to match the estimated value. The full PBPK model was applied in all simulations of the plasma concentration-time profiles of the drug in order to recover the shape of the concentration-time profiles.

Biliary clearance of leriglitazone was found to be relatively minor in sandwich-cultured hepatocytes with a CLint of 0.0005 L/min/mg protein (0.358 L/min/million hepatocytes). After scaling up the biliary clearance component accounted for a fraction of 19.5% of total clearance.

CYP2C8 and CYP3A4 were identified in vitro as the major contributing enzymes of leriglitazone with CYP2C8 mostly responsible for the formation of the main metabolite M3. Kinetic parameters of human CYP2C8 and CYP3A4 mediated metabolism of leriglitazone were studied in in vitro studies using recombinant CYP3A4 and CYP2C8 enzymes (ADMESCOPE, Finland). The fitted Km and Vmax values for CYP2C8 were 34.0 μM and the Vmax 2.27 pmol/min/pmol CYP based on M3 formation, and 13.7 μM and 1.60 pmol/min/pmol CYP based on leriglitazone depletion. As the disappearance of leriglitazone was very slow especially at high initial concentrations, the accuracy of the fit was not as good as based on M3 formation. The metabolism of leriglitazone by recombinant CYP3A4 did not saturate with the used initial concentrations. Thus, it was not possible to fit the kinetic parameters Km and Vmax. Based on these results, the contribution of CYP2C8 remains more significant (fraction metabolized around 70%) over the therapeutic range, i.e., up to 30 μM initial incubation concentration.

From the individual PK data on leriglitazone and M3 plasma concentration data, up to 51% of leriglitazone appears to be converted to M3 (Maximal M3: leriglitazone AUC ratio=0.51) and the metabolism of leriglitazone was split among CYP2C8 (63.7%), CYP3A4 (30%) and undefined metabolism (6%). After accounting for alternative clearance, namely biliary (f_{eBIL}=19.5%) and renal clearances (fe=5%), the simulated fraction metabolized f_{mCYP2C8} and f_{mCYP3A4} were 45% and 24%, respectively, and 6% for undefined metabolism. Intrinsic clearance values for CYP2C8 and CYP3A4 were calculated based on the estimated fm values and the reported mean CL/F value of 0.93 L/h from the phase 1 study (MAD part day 8).

Predictions of plasma concentration-time profiles and clearance were performed in the Simcyp Simulator using the default Sim-Healthy volunteer population, whose parameter values have been described previously. Howgate, E. M., et al., Xenobiotica Fate Foreign Compd. Biol. Syst. 36, 473-497 (2006); Inoue, S. et al., Xenobiotica Fate Foreign Compd. Biol. Syst. 36, 499-513 (2006). The trial design used for simulation of the plasma concentration-time profile of a single dose of leriglitazone in fasted and fed subjects was based on the phase 1 SAD results. Concentration-time profiles, using 10 virtual trials of 6 subjects (all male) aged 21 to 50 years receiving a single oral dose of 30, 90 or 270 mg under the fasted conditions and a single oral dose of 90 or 270 mg under fed conditions were simulated over a duration of 144 hours. Visual checks of the predicted concentration-time profiles were performed and the key PK parameters (AUC, Cₘₐₓ and Tₘₐₓ) were compared to those observed in the phase 1 study (Table 8).

### PBPK Model Verification

The PBPK model was verified by comparing the predicted concentration-time profiles with the observed phase 1 MAD data. The trial design used for simulation was based on multiple doses of 135 mg or 270 mg in fed healthy subjects. Concentration-time profiles, using 10 virtual trials of 8 healthy subjects (all male) aged 19 to 54 years receiving 135 mg or 270 mg daily dose orally for 8 days, were simulated under the fed conditions over a duration of 192 hours. The ka value estimated from the 90 mg dose (=0.89 h⁻¹) was used in the 135 mg simulation. The model was considered acceptable when the ratio of the predicted:observed parameter was between 0.8 and 1.25-fold.

### PBPK Model Application for Estimation of Starting Pediatric Doses

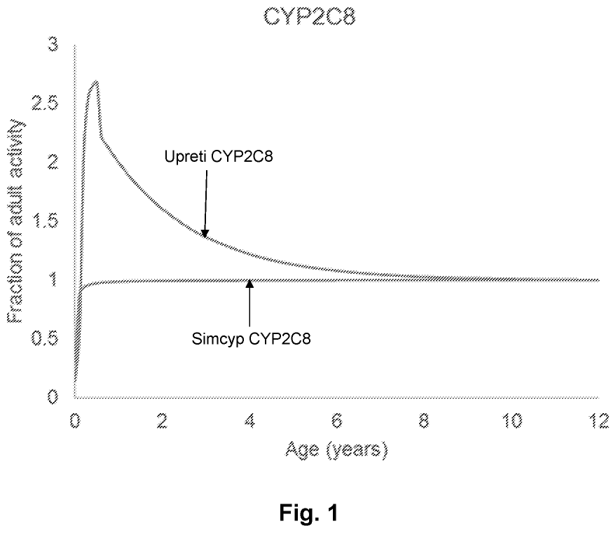

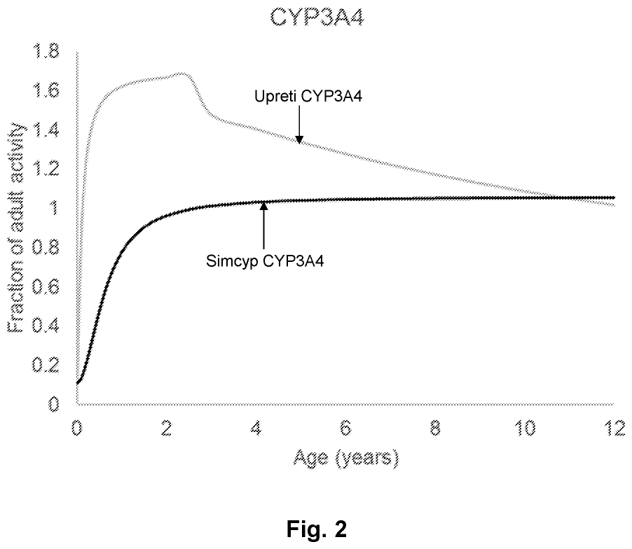

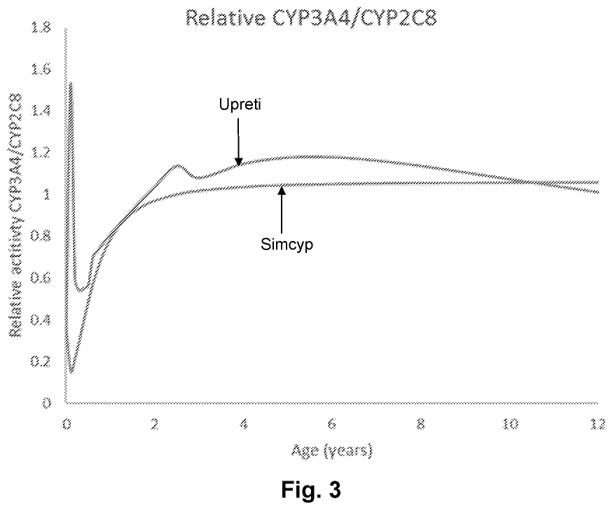

The ‘Simcyp Pediatric Simulator’ was used to model pharmacokinetic behavior of leriglitazone in neonates, infants, children and adolescents. The default Simcyp ontogeny functions for CYP3A4 and CYP2C8 as well as literature functions (Upreti, V. V. & Wahlstrom, J. L., J. Clin. Pharmacol. 56, 266-283 (2016)) were applied in separate simulations to account for the uncertainty in CYP ontogeny functions (FIG. 1, FIG. 2, and FIG. 3). The developed PBPK model in the fasted state was prospectively applied to estimate an appropriate starting dose for pediatric clinical evaluation, by matching a target systemic exposure of 160 μg·h/mL. To account for potential non-linearity issue with Cₘₐₓ, the developed adult drug models at the dose level of 30 mg or 90 mg were directly used, based on the range of simulated Cₘₐₓ values in the pediatric subjects.

The pediatric population was split into the following age bands, 0 to 1, 1 to 1.5, 1.5 to 2, >2 to 6, >6 to 12 and >12 to <18 years and multiple-dose simulations in the fasted state were undertaken to derive pediatric doses applying the default Simcyp as well as Upreti CYP ontogeny functions. The starting doses for pediatric clinical evaluation were projected based on body weight (BW). Allometric scaling was also applied to scale doses based on body size.

Concentration-time profiles, using 10 virtual trials of 10 pediatric subjects (50% female) in the age groups of 0 to 1, 1 to 1.5, 1.5 to 2, 2-<6, 6-<12 and 12-<18 years old, receiving respective daily doses of leriglitazone orally for 8 days, were simulated under the fasted conditions. The default Simcyp as well as Upreti CYP ontogeny functions assume the same relationship between enzyme activity and age in both males and females. Therefore, the simulation outcomes do not differ significantly between simulations assuming 100% male subjects versus 50% male.

PBPK Verification with Pediatric Patient Data

A phase 2 open-label clinical study in cALD with five recruited male pediatric patients aged 5-12 years is being conducted to assess the effects of leriglitazone treatment on disease progression prior to human stem cell transplant.

Observed plasma concentrations of leriglitazone from 5 patients have been used for the preliminary verification of the pediatric PBPK model: one in the 2-5 years age group, three in the 6-11 years age group as well as one patient above 12 years old who is in compassionate use (Table 3).

TABLE 3

| Patient | Age (years) | Age group | Weight (kg) |
|---|---|---|---|
| 1 | 5 | 2-5 y | 20 |
| 2 | 6 | 6-11 y | 27 |
| 3 | 9 | 6-11 y | 42 |
| 4 | 10 | 6-11 y | 36 |
| 5 | 12 | Above 12 y | 39 |

Following the first administration of leriglitazone HCl, blood samples were drawn at pre-dose, 2, 6, 12 and 24 h post-dose and, after 4 and 12-week treatment, at pre-dose and 2 h post-dose. The observed plasma concentrations after single or repeated dose were overlaid on the PBPK simulations using Upreti ontogeny for the corresponding age group. Visual checks were performed to confirm if values fell within the 95% Confidence Interval the confidence intervals of the predicted concentration-time profiles.

### Results

### Safety

All treatment-emergent adverse events (TEAEs) were of mild severity and resolved without sequelae by follow-up. There were no serious AEs reported. Overall, the percentage of subjects reporting TEAEs was similar after placebo and leriglitazone HCl administration (Table 4).

TABLE 4

| | | 135 mg | 270 mg |
|---|---|---|---|
| | Placebo | Leriglitazone | Leriglitazone |
| | N = 5 | N = 9 | N = 8 |
| SOC | E n (%) | E n (%) | E n (%) |
| Total | 12 4 (80) | 13 5 (56) | 16 8 (100) |
| Nervous system disorders | 5 2 (40) | 5 3 (33) | 3 3 (38) |
| Gastrointestinal disorders | 1 1 (20) | 2 1 (11) | 4 3 (38) |
| Musculoskeletal and | 1 1 (20) | 3 3 (33) | 1 1 (13) |
| connective tissue disorders | | | |
| General disorders and | 1 1 (20) | 1 1 (11) | 1 1 (13) |
| administration site conditions | | | |
| Respiratory, thoracic and | 1 1 (20) | | 3 3 (38) |
| mediastinal disorders | | | |
| Infections and infestations | 1 1 (20) | | 2 2 (25) |
| Skin and subcutaneous | 2 2 (40) | | |
| tissue disorders | | | |
| Eye disorders | | | 2 1 (13) |
| Metabolism and | | 1 1 (11) | |
| nutrition disorders | | | |
| Renal and urinary disorders | | 1 1 (11) | |

[symbol]

[symbol]

indicates data missing or illegible when filed

Therefore, single and multiple doses of leriglitazone HCl appear to be safe and well tolerated in healthy male subjects. No clinically significant changes from baseline were observed for clinical laboratory evaluations, vital signs, ECGs and physical examinations.

### Pharmacokinetics of Leriglitazone in Healthy Volunteers

The PK parameters of leriglitazone obtained in the SAD are shown in Table 5.

TABLE 5

| | | | | | | |
|---|---|---|---|---|---|---|
| | | 30 mg | 90 mg | 270 mg | | |
| | | fasted | fasted | fasted | 90 mg fed | 270 mg fed |
| Parameter | Statistics | n = 6 | n = 6 | n = 5 | n = 3 | n = 5 |

Leriglitazone

| | | | | | | |
|---|---|---|---|---|---|---|
| Cₘₐₓ (ng/ml) | Geometric | 1,695 (20.9) | 4,432 (9.5) | 13,407 | 3,171 (7.2) | 11,451 |
| | mean (CV %) | | | (23.9) | | (19.9) |
| Tₘₐₓ (h) | Median | 0,63 | 0.88 | 1.50 | 3.00 | 4.00 |
| AUC₀₋ₜ | Geometric: | 41,353 | 116,621 | 359,715 | 115,517 | 357,333 |
| (ng.h/ml) | mean (CV %) | (11.1) | (9.6) | (18.6) | (7.6) | (19.4) |
| AUC | Geometric | 44,166 (9.8) | 120,591 | 369,230 | 120,518 | 368,420 |
| (ng.h/mL) | mean (CV %) | | (9.9) | (18.2) | (7.0) | (18.9) |
| CL/F (L/h) | Geometric | 0.679 (9.0) | 0.746 (10.1) | 0.731 (16.2) | 0.747 (6.7) | 0.733 (17.8) |
| | mean (CV %) | | | | | |
| V_{z}/F (L) | Geometric | 23.2 (17,6) | 26.1 (9.4) | 24.0 (19.4) | 27.8 (12.8) | 24.9 (20.6) |
| | mean (CV %) | | | | | |
| t_{1/2} | Geometric | 23.6 (10.5) | 24.2 (8.1) | 22.7 (6.4) | 25.8 (6.8) | 23.5 (6.6) |
| | mean (CV %) | | | | | |

M3

| | | | | | | |
|---|---|---|---|---|---|---|
| Cₘₐₓ (ng/mL) | Geometric | 828 (13.7) | 2,553 (13.7) | 6,643 (17,9) | 1,982 (18.5) | 5,323 (19.8) |
| | mean (CV %) | | | | | |
| Tₘₐₓ (h) | Median | 0.63 | 0.63 | 1.50 | 5.00 | 4.00 |
| AUC₀₋ₜ | Geometric | 15,158 | 59,440 | 166,422 | 59,865 (8.8) | 154,719 |
| (ng.h/ml) | mean (CV %) | (23.3) | (18.1) | (22.9) | | (22.4) |
| AUC | Geometric | 17,574 | 61,835 | 169,880 | 62,215 (8.7) | 158,684 |
| (ng.h/mL) | mean (CV %) | (19.6) | (17.5) | (23.0) | | (22.8) |
| t_{1/2} (h) | Geometric | 22.1 (8.2) | 23.2 (11.3) | 21.6 (4.5) | 25.7 (5.1) | 22.7 (6.3) |
| | mean (CV %) | | | | | |

AUC = area under the concentration-time curve; CL/F = total body clearance; Cmax = maximum concentration; max = maximum; min = minimum; SAD = single ascending dose; t1/2 = half-life; Tmax = time to Cmax; Vz/F = volume of distribution
#For Tmax, the medlan (range) is presented instead of geometric mean (min-max).
*All concentrations are reported as total concentrations

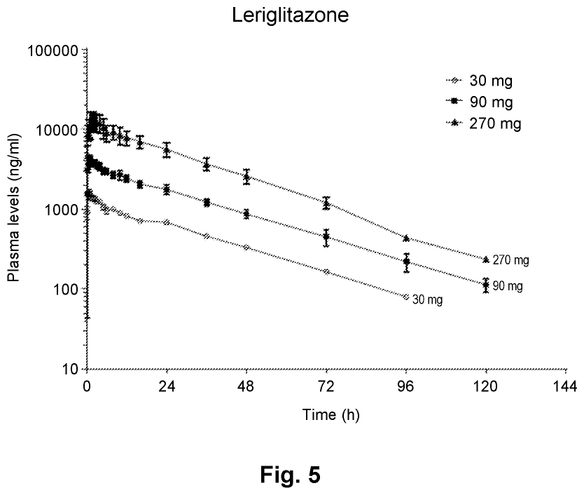

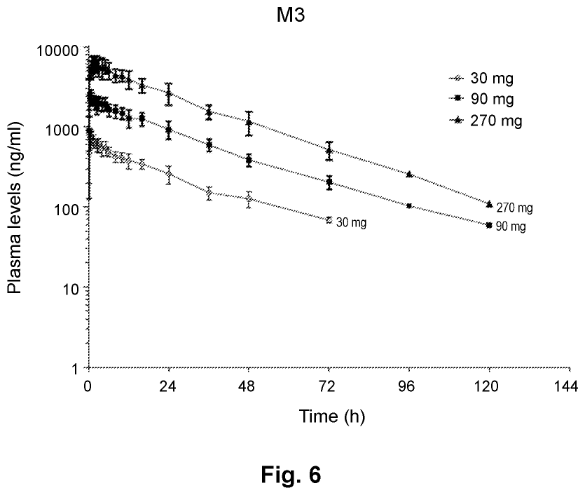

Under fasting conditions leriglitazone was rapidly absorbed with measurable concentrations of both leriglitazone and M3 in plasma at the first post-dose sampling time point (0.25 h) in all subjects at all tested doses (30, 90 and 270 mg). The time (Tₘₐₓ) to reach maximal plasma concentrations (Cₘₐₓ) ranged between 0.25 and 2.5 hours. After reaching a maximum, the concentrations of leriglitazone and M3 showed a steady decline. Half-lives were around 23-24 h for leriglitazone and 22-23 h for M3. In general, AUC increased proportionally with the dose. Combined individual leriglitazone and M3 plasma concentration-time profiles showed modest inter-subject variation within the same dose levels (FIG. 5 and FIG. 6). The food effect on leriglitazone absorption was also evaluated. Under fed conditions administration of leriglitazone resulted in a clear delay in the uptake of leriglitazone at all tested doses (90 and 270 mg). Tₘₐₓ of leriglitazone and M3 was observed at 3 to 6 hours with lower Cₘₐₓ values compared to the fasted state but the AUC was essentially not different from the fasting state. Therefore, food lowered the Cₘₐₓ and prolonged the Tₘₐₓ, but overall exposure was not affected.

Approximately 6.8% of the leriglitazone dose was excreted in urine over the 48-hour collection period as leriglitazone (6.5%) and M3 (0.3%), mostly in a conjugated state. Only a limited amount was excreted as free leriglitazone (<0.4%) or M3 (<0.01%). Food did not change the amount of leriglitazone and M3 excreted in urine.

The PK parameters obtained in the MAD are shown in Table 6.

TABLE 6

| | | Leriglitazone | | | | M3 | | | |
|---|---|---|---|---|---|---|---|---|---|
| | | 135 mg | 270 mg | 135 mg | 270 mg | 135 mg | 270 mg | 135 mg | 270 mg |
| | | MD | MD | MD | MD | MD | MD | MD | MD |
| | | Day 1 | Day 1 | Day 8 | Day 8 | Day 1 | Day 1 | Day 8 | Day 8 |
| Parameter | Statistics | n = 8 | n = 8 | n = 8 | n = 7 | n = 8 | n = 8 | n = 8 | n = 7 |
| Cₘₐₓ | Geometric | 5.429 | 10,007 | 9.496 | 16,990 | 2,468 | 5,362 | 4.148 | 8,032 |
| (ng/ml) | mean | (18.2) | (9.9) | (18.4) | (20.3) | (26.3) | (16.5) | (19.6) | (19.5) |
| | (CV %) | | | | | | | | |
| Tₘₐₓ (h) | Median | 3 | 3 | 4.00 | 4 | 3.5 | 3.5 | 2.75 | 2.5 |
| AUC₀₋ [symbol] | Geometric | 84,842 | 165,147 | 144,777 | 290,956 | 36,621 | 81,967 | 62,768 | 126,788 |
| (ng.h/ml) | mean | (39.6) | (13.4) | (21.8) | (22.3) | (30.1) | (19.6) | (25,2) | (20.7) |
| | (CV %) | | | | | | | | |
| CL/F (L./h) | Geometric | 1.59 (17.8) | 1.63 (14.4) | 8.932 | 6.928 | 3.69 (29.4) | 3.29 (26.4) | 2.15 (27.6) | 2.13 (25.9) |
| | mean | | | (19.8) | (13.9) | | | | |
| | (CV %) | | | | | | | | |
| V_{z}/F (L) | Geometric | 47.13 | 52.7 (25.3) | 27.7 (17.3) | 29.5 (15.7) | 111.4 | 102.8 | 63.5 (26.1) | 69.3 (25.7) |
| | mean | (24.3) | | | | (54.5) | (25.1) | | |
| | (CV %) | | | | | | | | |
| t [symbol] (h) | Geometric | | | 20.6 (7.3) | 22.1 (9.3} | | | 20.5 (9.9) | 22.8 (9.9) |
| | mean | | | | | | | | |
| | (CV %) | | | | | | | | |
| CSF C4h | Geometric | | | 188 (15.9) | 332 (13.4) | | | 13 (21.5} | 21 .4 (23.3) |
| (ng/mL) | mean | | | | | | | | |
| | (CV %) | | | | | | | | |
| Plasma C4h | Geometric | | | 88:7 (3.3) | 14,197 (29) | | | 3605 (9.8) | :5673 (34.2) |
| (ng/mL) | mean | | | | | | | | |
| | (CV %) | | | | | | | | |
| CSF/Plasma | Geometric | | | 2.1 (25.5) | 2.4 (35.4) | | | 0.4 (15.8) | 0.4 (43.3) |
| (%) | mean | | | | | | | | |
| | (CV %) | | | | | | | | |

AUC = area under the concentration-time curve; CL/F = total body clearance; Cmax = maximum concentration; CV = coefficient of variation, MAD = multiple ascending dose; max = maximum; MD = multiple dose, min = minimum; t1/2 = half-life; Tmax = time to Cmax; Vz/F = volume of distribution
*All concentrations are reported as total concentration [symbol]
indicates data missing or illegible when filed

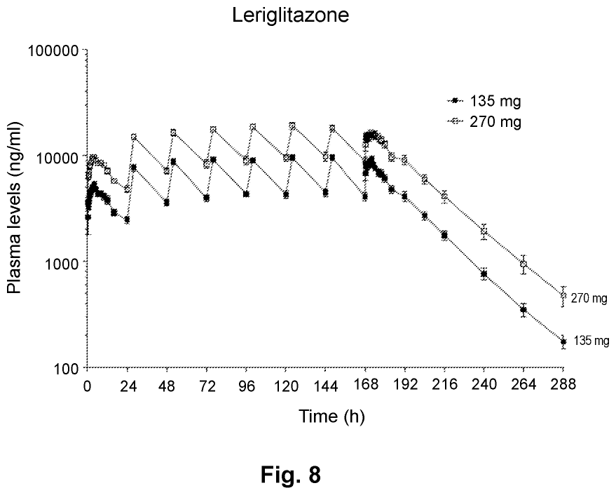

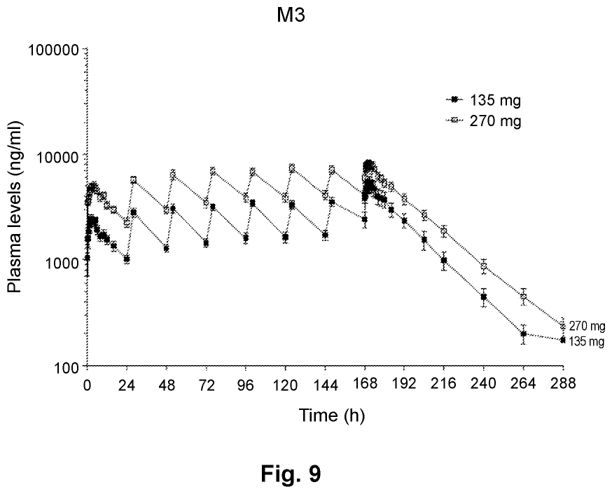

Plasma concentration-time profiles for leriglitazone and M3 following the first dose of 135 or 270 mg in fed conditions were similar to the profiles obtained in the SAD. Steady state was reached after 5 days. On day 8, the concentration vs. time profiles showed increased concentrations (FIG. 8 and FIG. 9). Both leriglitazone and M3 showed accumulation with RCₘₐₓ=1.7 for leriglitazone (both dose levels) and 1.57 to 1.69 for M3 (for 135 and 270 mg, respectively). RAUC ranged from 1.63 (M3 after 270 mg dose) to 1.81 (leriglitazone after 270 mg).

On day 8 at 4 hours post-dose, total CSF concentrations of leriglitazone and M3, respectively, were 188 and 13 ng/mL for 135 mg, and 332 and 22 ng/mL for 270 mg (Table 6). Thus, doubling the dose of leriglitazone nearly doubled the CSF concentration of leriglitazone (1.8-fold) and M3 (1.7-fold). Total leriglitazone concentrations in CSF represented 2.2-2.5% of total plasma concentrations whereas M3 only 0.4%, indicating brain penetration of leriglitazone was good while it was limited for M3.

### Brain Penetration of Leriglitazone in Rats and Humans

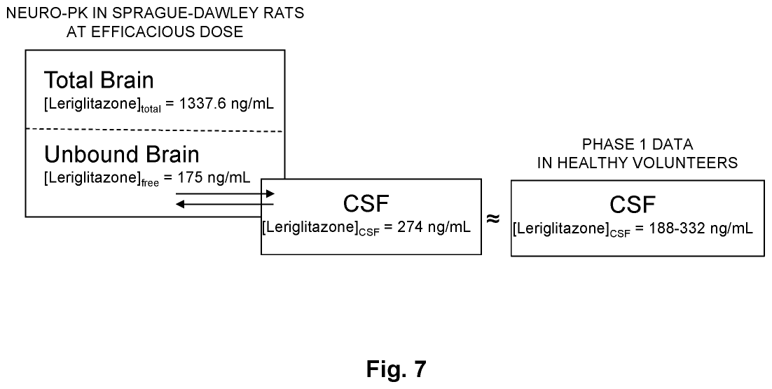

In a single dose neuro-pk study in rats, at the efficacious dose of 17 mg/kg, the unbound brain concentration of leriglitazone was 175 ng/ml similar to the measured CSF concentration of 274 ng/ml (FIG. 7). Given the validity of CSF as a surrogate marker for brain unbound concentration was proven in rats, CSF levels of leriglitazone were measured in Phase 1 study and confirmed to be similar as in rodents. Unbound brain to plasma ratio (Kpu,u) of leriglitazone as a measure of brain distribution of free drug was 0.55 in humans and 0.25 in rats (Table 7).

TABLE 7

| | | Total | Total | | | | |
|---|---|---|---|---|---|---|---|
| | | plasma | plasma | Total brain | Total brain | | Kpu, u |
| | | AUC | Cmax | AUC | Cmax | CSF Cmax | based |
| Species | Dose | (ng·.h/mil) | (ng/ml) | (ng·h/ml) | (ng/ml) | (ng/ml) | on CSF |
| rat | 17 mg/kg | 102173 | 21095 | 6582 | 1337.6 | 274 | 0,25 |
| human | 135 mg | 144550 | 9488 | | | 188 | 0.55 |
| human | 270 mg | 298662 | 17206 | | | 332 | 0.54 |

These results indicate that the drug exhibits good brain penetration, consisting with high passive permeability of the drug and as a result limited role for major brain efflux transporters such as P-gp and/or BCRP in brain penetration. These data also support the subsequent use of the PBPK model to predict starting pediatric doses of the drug and design the PK sampling scheme based on matching target systemic exposures in adults.

### Development and Verification of a PBPK Model for Leriglitazone

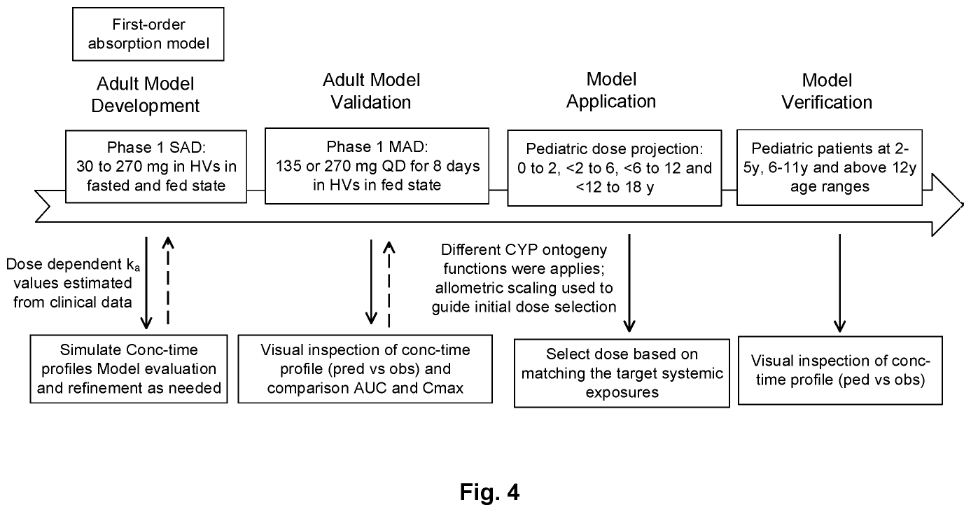

A PBPK model for leriglitazone was developed using the Simcyp software (FIG. 4, Table 1). The model incorporated CYP3A4 and CYP2C8-mediated metabolism as well as biliary clearance derived from in vitro data and the estimated fm_{CYP3A4}, fm_{CYP2C8} and f_{eBIL} were 24%, 45% and 19.5%, respectively.

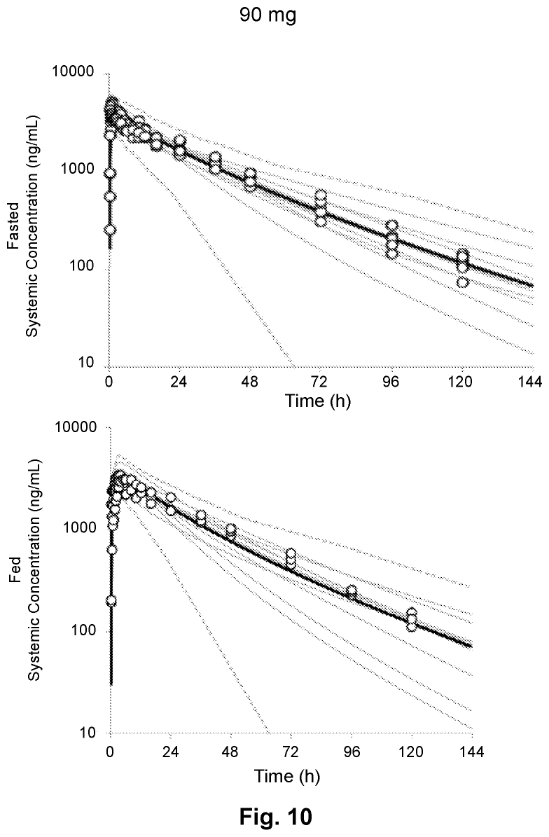

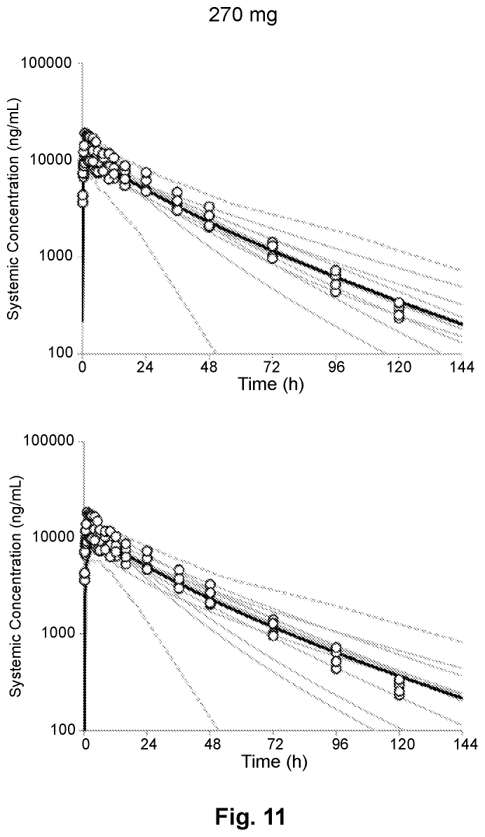

During model development, the SAD data were used as the model training dataset and the MAD data as the model verification dataset. Application of the developed model recovered the systemic exposure following the single-dose administration of 90 to 270 mg in either fed or fasted state in HV. A comparison of simulated and observed plasma concentration-time profiles for leriglitazone following a single 90 or 270 mg oral dose in fasted and fed conditions are shown in FIG. 10 and FIG. 11. The concentration-time profiles for the single 30 mg oral dose in fasted conditions were also simulated (Data not shown). The model adequately recovered the clinical data since the majority of the observed individual data points were within the 5% to 95% percentiles and at least one trial recovered the observed mean data up to 120 hours post dose. The simulated AUC and Cₘₐₓ values were within 0.80 to 1.15-fold of the observed values (Table 8).

TABLE 8

| | | PK | | | |
|---|---|---|---|---|---|
| Dose | Fasted/Fed | Parameter | Predicted | Observed | Pred/Obs |
| 30 mg | Fasted | Cmax (ng/ml) | 1828 | 1695 | 1.08 |
| | | AUC (ng/mlh) | 35514 | 44166 | 0.80 |
| | | Tmax (h) | 0.6 | 0.63 | 0.89 |
| 90 mg | Fasted | Cmax (ng/ml) | 5089 | 4432 | 1.15 |
| | | AUC (ng/ml h) | 106507 | 120591 | 0.88 |
| | | Tmax (b) | 0.71 | 0.88 | 0.81 |
| | Fed | Cmax (ng/ml) | 3586 | 3171 | 1.13 |
| | | AUC (ng/ml-h) | 103429 | 120518 | 0.86 |
| | | Tmax (h) | 2.5 | 3 | 0.85 |
| 270 mg | Fasted | Cmax (ng/ml) | 12731 | 13407 | 0.95 |
| | | AUC (ng/mlh) | 319345 | 369230 | 0.86 |
| | | Tmax (h) | 1.3 | 1.5 | 0,90 |
| | Fed | Cmax (ng/ml) | 9478 | 11451 | 0.83 |
| | | AUC (ng/ml-h) | 309776 | 368421 | 0.84 |
| | | Tmax (b) | 4.6 | 4 | 1.16 |

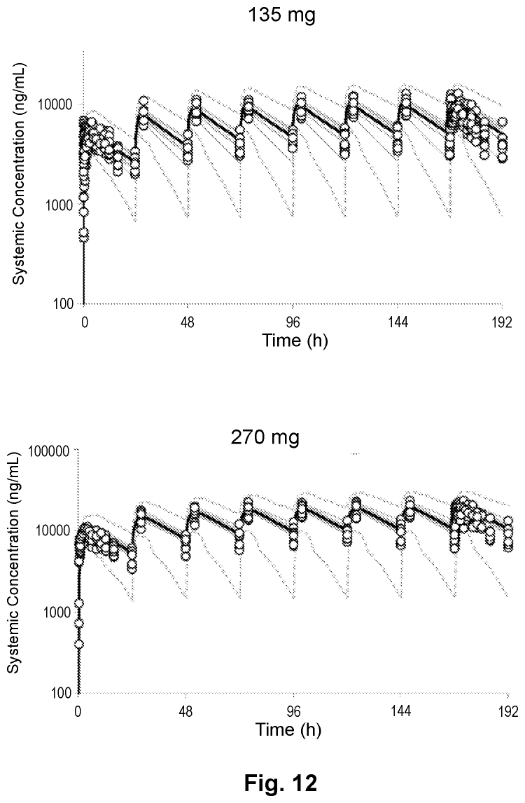

As model verification, subsequent simulations also recovered the systemic exposure following the multiple-dose administration of 135 mg to 270 mg in HV in the fed state. The model adequately recovered the clinical data since the majority of the observed data points were within the 5% to 95% percentiles and at least one trial recovered the observed mean data (FIG. 12). The simulated steady-state AUC and Cₘₐₓ values were within 1.04 to 1.14-fold of the observed values (Table 9).

TABLE 9

| Dose | Timepoint | PK Parameter | Predicted | Observed | Pred/Obs |
|---|---|---|---|---|---|
| 135 mg | Day 1 | Cmax (ng/ml) | 5551 | 5487 | 1.01 |
| | | AUC (ng/mlh) | 90449 | 85102 | 1.06 |
| | | Tmax (h) | 2.8 | 3.0 | 0.93 |
| | Day 8 | Cmax (ng/ml) | 9857 | 9488 | 1.04 |
| | | AUC (ng/mlh) | 164106 | 144550 | 1.14 |
| | | Tmax (h) | 2.4 | 4 | 0.60 |
| 270 mg | Day 1 | Cmax (ng/ml) | 9931 | 10007 | 0.99 |
| | | AUC (ng/ml-h) | 175804 | 165147 | 1.06 |
| | | Tmax (h) | 5 | 3 | 1.06 |
| | Day 8 | Cmax (ng/ml) | 18269 | 17206 | 1.06 |
| | | AUC (ng/ml-h) | 328466 | 298662 | 1.10 |
| | | Tmax (h) | 3.9 | 4 | 0.97 |

### Application of the PBPK Model for Estimation of Starting Pediatric Doses

The PBPK model was prospectively applied to estimate an appropriate starting dose for pediatric clinical evaluation, by matching the target systemic exposure of 160-μg·h/mL, based on the preclinical pharmacology and safety evaluation together with phase 1 data. This target systemic exposure is equivalent to an adult dose of 150 mg QD, based on linear extrapolation from the data obtained at 135 mg QD. The default Simcyp as well as Upreti ontogeny functions for CYP3A4 and CYP2C8 were applied in separate simulations.

Multiple-dose simulations for 8 days (fasted) to match the target systemic exposure in adult patients receiving 150 mg were undertaken to derive pediatric doses. When applying the default Simcyp ontogeny functions the predicted doses were 1.9, 1.9, 2.0, 2.2, 2.2 and 2.1 mg/kg QD whereas when applying the Upreti ontogeny functions the doses were 2.4, 2.3, 2.3, 2.4, 2.3 and 2.1 mg/kg QD for the age groups of 0-1, 1-1.5, 1.5-2,2-<6, 6-<12 and 12-<18 years old, respectively (Table 10).

TABLE 10

| | | Upreti ontogeny | | | Simcyp default ontogeny | | | |
|---|---|---|---|---|---|---|---|---|
| | Median BW | | Cmax, | AUCO-24 h, | | Cmax, | AUCO-24 h, | |
| | in the | | Day 8 | Day 8 | | Day 8 | Day 8 | Allometric* |
| | virtual | Dose | (ng/ml) | (ng/ml·h) | Dose | (ng/ml) | (ng/ml·h) | Dose |
| Age Range | population | (mg/kg | Geometric | Geometric | (mg/kg | Geometric | Geometric | (mg/kg |
| (y) | (Kg) | QD) | Mean | Mean | QD) | Mean | Mean | QD) |
| 0-1 | 7 | 2.4 | 14540 | 163377 | 1.9 | 12816 | 161160 | 3.5 |
| | | | 90% CI: | 90% CI: | | 90% CI: | 90% CI: | |
| | | | 14071 - | 151704 - | | 12342 - | 149148 - | |
| | | | 15024 | 175949 | | 13308 | 174140 | |
| 1-1.5 | 10.8 | 2.3 | 13870 | 159134 | | 12457 | 155757 | |
| | | | 90% CI: | 90% CI: | 1.9 | 90% CI: | 90% CI: | 3.1 |
| | | | 13418 - | 148157 - | | 12020 ~ | 144888 - | |
| | | | 14337 | 170925 | | 12910 | 167441 | |
| 1.5-2 | 12.2 | 2.3 | 13677 | 159941 | | 12842 | 162536 | |
| | | | 90% CI: | 90% CI: | 2.0 | 90% CI: | 90% CI: | 3.0 |
| | | | 13243 - | 149454 - | | 12412 - | 151917 - | |
| | | | 14125 | 171164 | | 13288 | 173897 | |
| 2-6 | 16 | 2.4 | 13369 | 157738 | | 12975 | 162534 | |
| | | | 90% CI: | 90% CI: | 2.2 | 90% Ci: | 90% CI: | 2.8 |
| | | | 12957- | 147743- | | 12545- | 152023- | |
| | | | 13793 | 168408 | | 13418 | 173771 | |
| >6-12 | 28 | 2.3 | 12419 | 157850 | | 12157 | 157606 | |
| | | | 90% CI: | 90% CI: | 2.2 | 90% CI: | 90% CI: | 2.4 |
| | | | 11977- | 146255- | | 11709- | 145889- | |
| | | | 12878 | 170365 | | 12622 | 170264 | |
| >12-17 | 51 | 2.1 | 12504 | 164327 | | 12424 | 162474 | |
| | | | 90% CI: | 90% CI: | 2.1 | 90% CI: | 90% CI: | 2.1 |
| | | | 12023- | 152075- | | 11955- | 150462- | |
| | | | 13004 | 177566 | | 12911 | 175444 | |

·Pediatric Dese : Adult Dose × (BWp/BWa)^{0.75}

Verification of the PBPK Model with Pediatric Patient Data

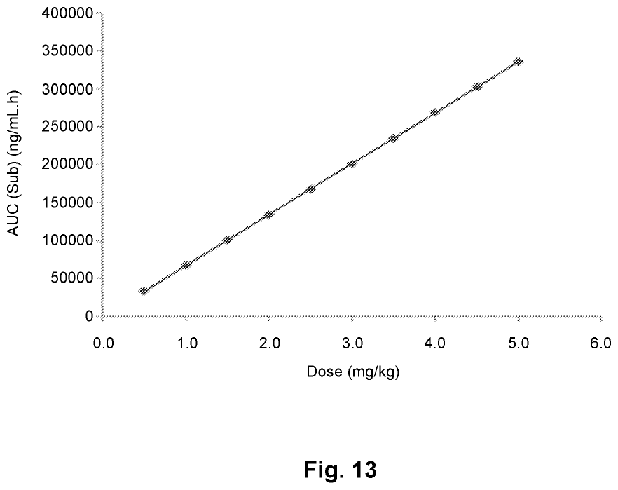

An open-label clinical study in cALD is ongoing to assess the effects of leriglitazone treatment on the progression of early cerebral lesions in male pediatric patients aged 2-12 years. The target exposure for this clinical study was set at 170 μg·h/mL slightly above the initially proposed exposure of 160 μg·h/mL, based on the preclinical pharmacology and safety evaluation together with phase 1 data. Thus, the pediatric starting doses for this clinical study were linearly extrapolated from the ones derived from the pediatric PBPK model for a systemic exposure of 160 μg·h/mL, as the simulated drug exposure is dose-proportional within the different pediatric age groups (FIG. 13).

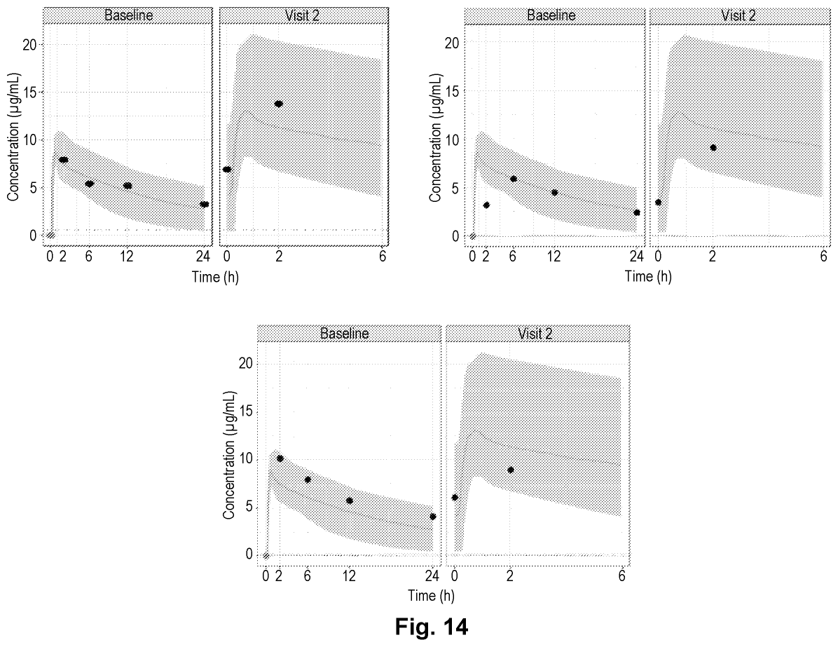

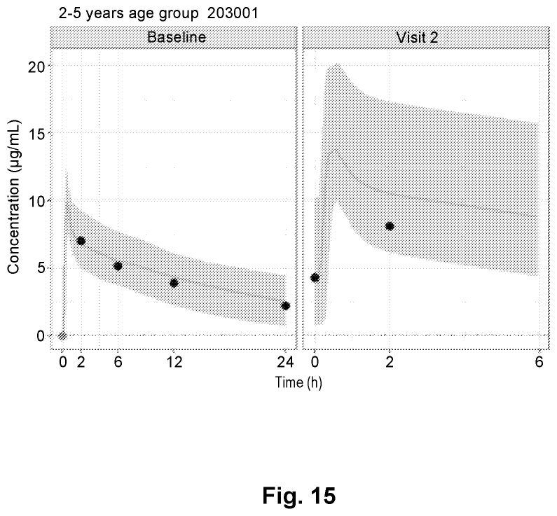

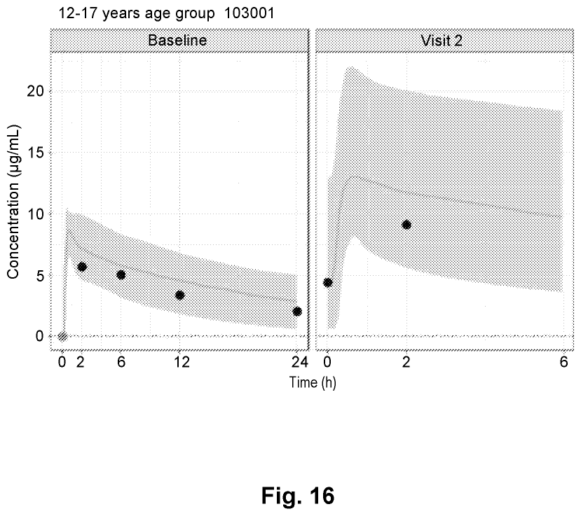

Data from five cALD patients from 5 to 12 years old were used to verify the estimation of starting pediatric dose with real pediatric patient data (Table 3). The observed plasma concentrations after single dose (baseline) or repeated dose (visit 2) were overlaid on the PBPK simulations using Upreti ontogeny for the corresponding age group. The observed data points are contained nicely within the 95% Confidence Interval (FIG. 14, FIG. 15, and FIG. 16).

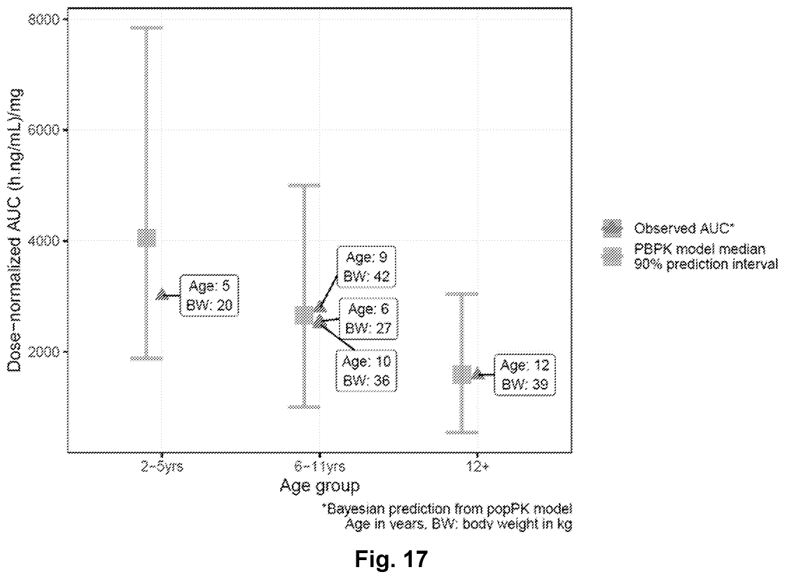

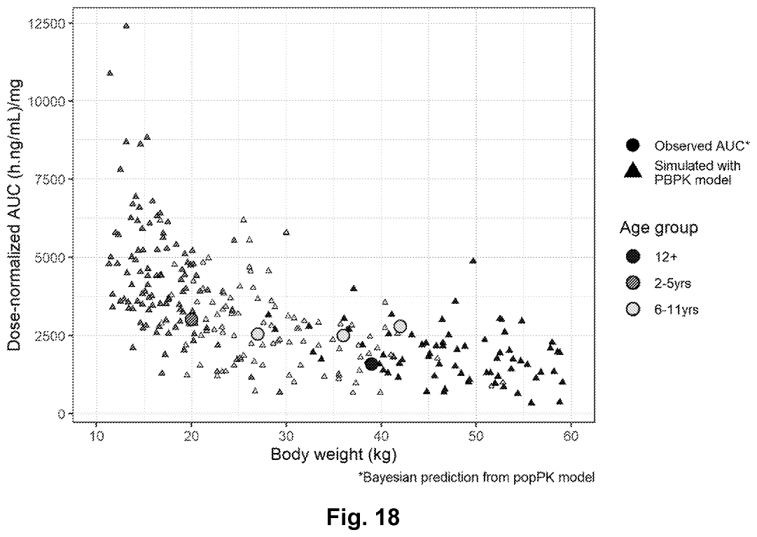

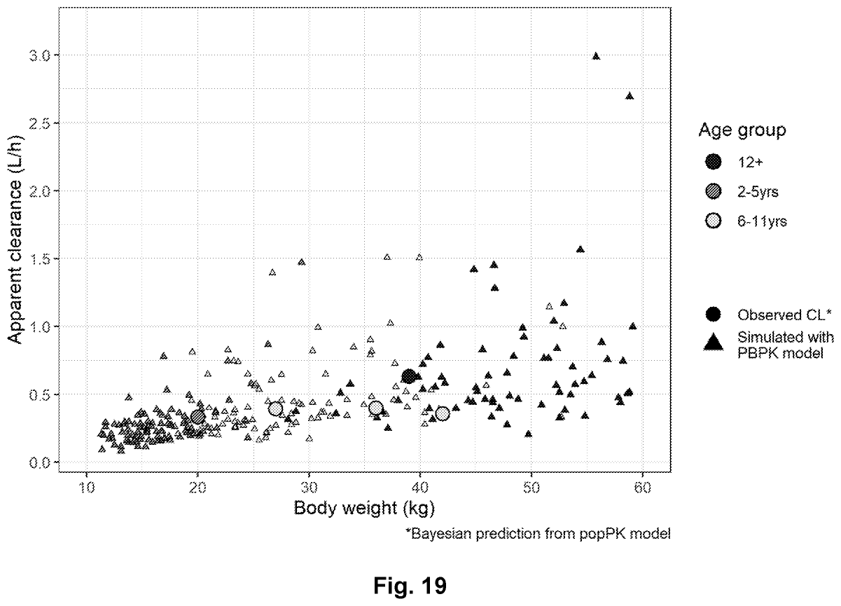

To further qualify the PBPK model, Individual Bayesian predictions of CL/F and AUC at steady-state (derived as: F×DOSE/CL) were obtained from the observed concentration measurements in the five pediatric patients using a population PK (popPK) model of leriglitazone developed in adults and compared to the corresponding parameters from the PBPK model across the age range of interest (see supplemental material for a brief description of the model). The Bayesian predicted parameters compare favorably with the PBPK simulations (FIG. 17, FIG. 18, and FIG. 19)

### Conclusion

A PBPK model was constructed to predict exposure and estimate starting doses in all pediatric age population and was verified by confirming that the predicted PK profiles were very similar to the phase 1 profiles. From the adult PBPK model, a pediatric PBPK model was developed using extensive libraries on pediatric demography, developmental physiology and biochemistry. Johnson, T. N. et al., Drug Metab. Dispos. Biol. Fate Chem. 44, 1090-1098 (2016). The predictive value of the pediatric Simcyp model has been evaluated for a number of drugs in neonates, infants, children, and adolescents. Johnson, T. N. et al., Paediatr. Anaesth. 21, 291-301 (2011). Neither PPARγ nor biliary clearance ontogeny functions were applied since their maturation seems to be rapid in pediatrics reaching adult activity very soon Johnson, T. N. et al., Drug Metab. Dispos. Biol. Fate Chem. 44, 1090-1098 (2016); Beck, F. et al. Proc. Biol. Sci. 247, 83-87 (1992). Conversely, the fraction metabolized f_{mCYP2C8} and f_{mCYP3A4} values used to develop the adult model are important as these are the key enzymes responsible for leriglitazone metabolism. Human clinical drug-drug interaction (DDI) data are not available yet to quantitatively estimate the overall fraction metabolised (fm) by each enzyme and the fm_{CYP2C8} and fm_{CYP3A4} values are based on the AUC-M3 to AUC-leriglitazone ratio and in vitro study using recombinant enzymes which could represent some limitations in the model. Although kinetic parameters could not be calculated due to the lack of saturation of the recombinant CYP3A4, both approaches converge in similar fm and are acceptable as an initial fm estimation for a population where the difference in CYP ontogeny is not relevant. The fm values used to develop the leriglitazone model in adults are important if there is a big difference in how the main enzymes CYP2C8 and CYP3A4 develop with age. For both the Simcyp default (Johnson, T. N. et al., Drug Metab. Dispos. Biol. Fate Chem. 44, 1090-1098 (2016)) and literature CYP ontogeny (Upreti, V. V. et al., J. Clin. Pharmacol. 56, 266-283 (2016)) profiles, the two enzymes in pediatric subjects above 2 years are either fully developed (Simcyp) or develop in parallel (Upreti) whereas below 2 years their activity differs significantly and the fm miss-assignment would be more critical. Indeed, application of the Simcyp default ontogeny led to slightly lower predicted pediatric doses since it assumes lower CYP2C8 and CYP3A4 activity, especially below 2 years (FIG. 1, FIG. 2, and FIG. 3). On the contrary, application of allometric scaling leads to over-prediction of the doses, which is evident in all age group below 6 years. Therefore, the starting pediatric doses for clinical studies were selected based on the Upreti ontogeny since it accounts for higher CYP activity being a more conservative approach. A preliminary verification of this pediatric PBPK model has been allowed by comparing the simulations with data from five pediatric cALD patients with very encouraging results. Additionally, Bayesian predicted parameters for the five pediatric cALD patients by using popPK model compare favorably with the PBPK simulations and serve as a verification of the PBPK model for pediatric use.

This study shows that leriglitazone, unlike other PPARγ agonists, presents an adequate PK to reach CNS target exposure and is well set to become the first PPARγ agonist used in neurodegenerative and neuroinflammatory indications. The PBPK model helped to gain understanding of the processes affecting leriglitazone PK, informed pediatric clinical development and starting doses and will be further used to support regulatory submissions and labeling including DDI.

### Example 2

Population Pharmacokinetic Modelling of Leriglitazone HCl in Healthy Volunteers and Patients with Neuro-Degenerative Disorders

### Clinical Studies Included:

MT-1-01 (healthy volunteers), MT-2-01 (adult men with adrenomyeloneuropathy (AMN), MT-2-03 (men, women≥12 years with Friedreich's Ataxia).

### Data

A total of 2140 valid plasma concentration measurements from 130 individuals were used in the analysis. Full PK profiles were available only from MT-1-01. In MT-2-03 only pre-dose (trough) concentrations were taken at multiple visits, while in MT-2-01 also sparse post-dose samples were taken. Less than 1% of all samples were below the limit of quantification.

### Methods

The following key steps were taken in the analysis:

1. Exploratory graphical analysis of concentration-time and covariate data.

2. Structural model-development using only data from healthy volunteers (MT-1-01) and inclusion of key structural covariates (food, body weight).

3. Re-estimation of parameters on the full analysis dataset (MT-1-01, MT-2-01, MT-2-03). Finalization of base model.

4. Stepwise automatic covariate modeling (SCM) using the base model.

5. Refinement of final model including significant covariates.

6. Qualification of final model (visual predictive check).

7. Simulations.

NONMEM v7.5 was used for the population modelling. The RxODE package (v0.9.2) for R (v4.0.2) was used for the simulations.

With respect to the covariate model development, a fixed allometric exponent of 0.75 for clearance and inter-compartment flow (Q) was used while an exponent of 1.0 was used for the relationship between volume and body weight.

Automated stepwise covariate modeling (SCM) using a forward addition/backward deletion procedure as implemented in PsN, Karlsson and Savic, C/in Pharmacol Ther 82:17-20 (2008), was applied to the base model on the complete analysis dataset. To improve the computation time of the SCM, PsN generates a linearized form of the model applying a Taylor-transformation on which then the covariate function is applied. Anderson et al., Annu Rev Pharmacol Toxicol 48:303-332 (2008). The final model from the SCM procedure was then rerun in its original, untransformed form.

The following covariates were tested in the model for their association with CL and relative bioavailability (F):
BMI
Height
Age
Ethnicity (Hispanic yes/no)
Sex

Laboratory variables: aspartate transaminase (AST), alanine transaminase (ALT), bilirubin, albumin, creatine clearance (Cockcroft-Gault)

At first only the covariate values at baseline were tested. In case of significance and inclusion in the model, the model was re-executed using the time-varying covariate value.

For continuous covariates linear or power functions were tested, as shown in the following equations:

$${P}_{i} = {P}_{TV} \times ( 1 + θ \times ( {COVAR}_{i} - {MED}_{COVAR} ) ) ; linear function$$

$${P}_{i} = {P}_{TV} \times {( \frac{{COVAR}_{i}}{{MED}_{COVAR}} )}^{θ} ; power function$$

Where Pᵢ is the parameter estimate for the iₜₕ patient with the value of the covariate (COVARᵢ) centered or scaled to the typical median of the covariate (MED_{COVAR}). θ is the slope (exponent) of the parameter-covariate relationship.

A dichotomous categorical covariate was included as shown below:

$${P}_{i} = {P}_{TV} \times ( 1 + θ \times CATCOVAR )$$

Where θ is the coefficient of the parameter-covariate relationship. CATCOVAR takes values of 0 or 1 for the state of the covariate value (absent/present) in a particular individual.

Forward covariate selection was performed using a p-value of p<0.05 (χ2p=0.05,v=1=3.84) as the selection criterion. Subsequently, backwards deletion was performed using a p-value of p<0.01 (χ2p=0.01,v=1=6.63) as the selection criterion.

The values of the coefficients for AGE, SEX, BW, BMI, and FOOD of Formula I are parameters of the model that quantitate and explain the variability of leriglitazone exposure in patients. They are estimated by fitting the model to the clinical data by means of nonlinear mixed-effects modelling. See, e.g., Mould and Upton, CPT. Pharmacometrics & Systems Pharmacology (2012) 1, e6; doi:10.1038/psp.2012.4; Mould and Upton, CPT. Pharmacometrics & Systems Pharmacology (2013) 2, e38; doi:10.1038/psp.2013.14; Mould and Upton, CPT Pharmacometrics Syst. Pharmacol. (2014) 3, e88; doi:10.1038/psp.2013.71

### Results

### Modelling

A two-compartment model with first-order absorption and linear elimination provided good fit to the available data. The typical rate of absorption was fast when leriglitazone was taken without food (5.2 h⁻¹), however was reduced by 82.9% when taken with food. Furthermore, food reduced the bioavailability by 4.8%. Body weight was included in the model as a covariate on clearance, inter-compartment flow and volume terms by means of allometric scaling with fixed exponents.

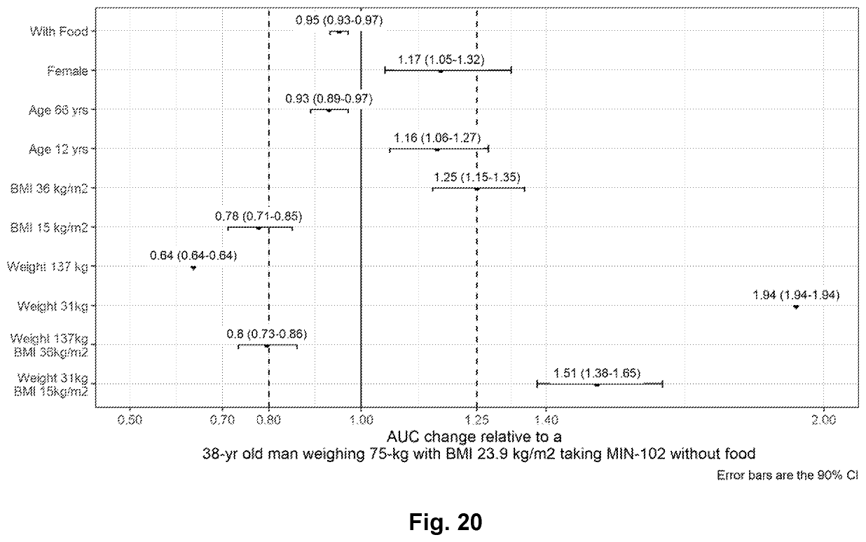

Further significant covariates were identified in an automatic covariate search including a stepwise forward search (significance criterion p<0.05) and a subsequent backward deletion step with a stricter criterion (p<0.01). Age was significantly associated with clearance (the younger the lower), as was sex (14.7% lower CL in women). Body mass index (BMI) was significantly correlated with (relative) bioavailability (larger values with higher BMI). FIG. 20 illustrates the effect of the covariates on AUC. (MIN-102 refers to leriglitazone).

### Simulations

Simulations were conducted comparing concentration-time profiles predicted with the popPK model to predictions obtained with a previously developed physiologically-based PK (PBPK) model. See EXAMPLE 1. Both models produced comparable results.

In addition, the model was used to simulate the concentration-time profile of children between 5 to 12 years and the resulting predictions matched closely with actual observation obtained in the ongoing pediatric study MT-2-02 of patients suffering from cerebral adrenoleukodystrophy (cALD).

### Conclusions

The pharmacokinetics of leriglitazone was generally well behaved, showing fast absorption (in fasted state), low variability, dose-proportional exposure and no indication of non-linearity of elimination over time. These favorable characteristics allowed the description of the data with a relatively simple 2-compartment population PK model. There was no indication of differences in PK between patients (Friedreich's Ataxia or AMN) and healthy volunteers, such that the estimation of model parameters on the joint dataset was straightforward. The single most important factor influencing the shape of the PK profile (though not the AUC) was food. Taking leriglitazone in the fed state reduced the absorption rate by 82.9% leading to a lower and later Cmax. The impact on bioavailability (and thus AUC), however, was minimal (<5%) and hence no specific dosing recommendation with respect to prandial state is necessary.

Body weight was implemented in the model as an influential factor on clearance terms (CL and Q) and volume terms (V1 and V2) according to the principle of allometric scaling. It is now well accepted in clinical pharmacology that these parameters typically scale with body size and the allometric exponents of 0.75 and 1.0 for clearance and volume, respectively, have been established. Using these theoretical exponents as fixed rather than estimating them is particularly useful when using the model for extrapolations to smaller individuals, i.e. children—that did not form part of the analysis dataset. Although body weight increased in patients in the active treatment group, using the actual weight at each visit instead of weight at baseline provided no advantage in model fit. The increase in weight is a known side effect of leriglitazone due to fluid retention in tissues, i.e. edema, and as long as it doesn't affect cardiac output or metabolic capacity in the liver this change has little relevance on the clearance of leriglitazone.

Additional covariates were only evaluated on CL and F. For one because the interindividual variability in the central volume could not be estimated properly, but also because the key PK parameter for clinical decision-making is AUC, which is determined by CL and F. The automated covariate search resulted in a significant association of F with BMI (higher F for higher BMI), CL with age (lower CL at younger age) and CL with sex (lower CL in females).

It should be noted that F is not a “clean” parameter and, in absence of intravenous data, it is not possible to determine its absolute value but only its variability relative to the typical value of 1. Moreover, bioavailability is a composite parameter that not only represents the fraction of drug absorbed from the gut but also the fraction that is not eliminated during the first pass through the liver. Therefore, the relationship with BMI cannot be attributed to a particular physiological process and should be seen as an empirical finding. For all practical purposes regarding dosing, it is anyway the combination of CL and F (aka “oral” clearance or CL/F) that determines steady-state exposure.

The effect of age on CL, although significant, was rather limited and given the curvature of the power function, it is more relevant at the younger ages than at older ages. As the forest plot illustrated the AUC may be 16 higher in a 12-year-old kid compared to the 38-year-old typical patient—all other covariates, including weight and BMI, kept equal. Simulations that compared the model predictions to actual observations in children younger than 12 years (MT-2-02) indicated that the age effect may not be very pronounced below 12 years. However, at this point the comparison could only be made with 4 patients between 5 and 12 years. An update of the model once all MT-2-02 PK data will be available may bring clarity on the actual relevance of this covariate.

### VI. Glossary

| | Difference in OFV between two |
|---|---|
| Δ OFV | competing hierarchical models |
| ABC | ATP binding cassette |
| ADDL | Additional doses (NONMEM data file item) |
| CALD | Cerebral adrenoleukodystrophy |
| ALD | Adrenoleukodystrophy |
| ALDP | ALD protein |
| AMC | Amsterdam Medical Centre |
| AMN | Adrenomyeloneuropathy |
| AUC | Area under the concentration-time curve |
| AST | Aspartate transaminase |
| ALT | Alanine transaminase |
| b.i.d. | Bis in die, twice a day |
| BLQ | Below the limit of quantification |
| BMI | Body mass index |
| BSA | Body surface area |
| CSF | Cerebrospinal fluid |
| CNS | Central Nervous System |
| CL | Clearance |
| CV | Coefficient of variation |
| CWRES | Conditionally weighted residuals |
| DBL | Database lock |
| DV | Dependent variable (NONMEM data file item) |
| EMA | European Medicines Agency |
| ELOVL 1 | Elongation of very long chain fatty acids protein 1 |
| FDA | U.S. Food and Drug Administration |
| F | Bioavailability |
| FE | Food effect |
| FOCE | First order conditional estimation |
| FRDA | Friedreich’s ataxia |
| GAM | Generalized additive model |
| GOF | Goodness of fit |
| IBW | Ideal body weight |
| II | Inter-dose interval (NONMEM data file item) |
| IIV | Inter-individual variability |
| IOV | Inter-occasion variability |
| IWRES | Individually weighted residuals |
| KA | Absorption rate constant |
| LBW | Lean body weight |
| LTBS | log-transform both sides |
| MAD | Multiple ascending dose |
| M&S | Modelling and simulation |
| NHANES | National health and nutrition examination survey |
| NONMEM | Non-linear Mixed Effects Model |
| NPC | Numerical predictive check |
| OFV | Objective function value |
| PBPK | Physiologically-based pharmacokinetics |
| PK | Pharmacokinetic(s) |
| PPC | Posterior predictive check |
| PPAR | Peroxisome proliferator-activated receptor |
| PRED | Population prediction |
| PSN | Perl speaks NONMEM |
| QC | Quality control |
| Q | Intercompartment flow |
| q.d. | quaque die, once daily |
| RSE | Relative standard error |
| SAD | Single ascending dose |
| SCM | Stepwise covariate modelling |
| SIR | Sampling importance resampling |
| TDM | Therapeutic drug monitoring |
| T2D | Type 2 diabetes |
| TSLD | Time since last dose |
| V | Volume of distribution |
| VLCFA | Very light chain fatty acids |
| VPC | Visual predictive check |
| WT | Body weight |
| X-ALD | X-linked adrenoleukodystrophy |

### VII. Particular Embodiments

The disclosure also provides the following particular embodiments.

Embodiment 1. A method of treating a disease or disorder in a patient in need thereof, the method comprising administering 5-[[4-[2-[5-(1-hydroxyethyl)pyridin-2-yl]ethoxy]phenyl]methyl]-1,3-thiazolidine-2,4-dione (leriglitazone), or a pharmaceutically acceptable salt thereof, to the patient, wherein the initial dose of leriglitazone, or a pharmaceutically acceptable salt thereof, in mg, is determined according to Formula I:

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 265 μg·h/mL of leriglitazone.

Embodiment 2. The method of Embodiment 1, wherein BW_{coef} is 0.75.

Embodiment 3. The method of Embodiments 1 or 2, wherein AGE_{coef} is 0.131.

Embodiment 4. The method of any one of Embodiments 1-3, wherein SEX_{coef} is 0.147.

Embodiment 5. The method of any one of Embodiments 1-4, wherein BMI_{coef} is 0.541.

Embodiment 6. The method of any one of Embodiments 1-5, wherein FOOD_{coef}=0.0477.

Embodiment 7. The method of any one of Embodiments 1-6, wherein the disease or disorder is a CNS disease or disorder.

Embodiment 8. The method of any one of Embodiments 1-6, wherein the disease or disorder is a lung disease or disorder.

Embodiment 9. The method of any one of Embodiments 1-8, wherein the target AUC_{target} is 100 to 135 μg·h/mL.

Embodiment 10. The method of any one of Embodiments 1-8, wherein the target AUC_{target} is 150±20% μg·h/mL.

Embodiment 11. The method of any one of Embodiments 1-8, wherein the target AUC_{target} is 170±20% μg·h/mL.

Embodiment 12. The method of any one of Embodiments 1-8, wherein the target AUC_{target} is 190±20% μg·h/mL.

Embodiment 13. The method of any one of Embodiments 1-8, wherein the target AUC_{target} is 200±20% μg·h/mL.

Embodiment 14. The method of any one of Embodiments 1-8, wherein the target AUC_{target} is 220±20% μg·h/mL.

Embodiment 15. The method of any one of Embodiments 1-6, wherein the disease or disorder is a liver disease or disorder.

Embodiment 16. The method of Embodiment 15, wherein the AUC_{target} is 50±20% μg·h/mL.

Embodiment 17. The method of any one of Embodiments 1-11, wherein the patient is a pediatric patient.

Embodiment 18. The method of any one of Embodiments 1-16, wherein the patient is an adult patient.

Embodiment 19. The method of any one of Embodiments 1-18, wherein leriglitazone is administered.

Embodiment 20. The method of Embodiments 1-18, wherein leriglitazone HCl is administered.

Embodiment 21. Leriglitazone, or a pharmaceutically acceptable salt thereof, for use in treating a disease or disorder in a patient in need thereof, wherein the initial dose of leriglitazone, or a pharmaceutically acceptable salt thereof, in mg, to be administered is determined according to Formula I.

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 265 μg·h/mL of leriglitazone.

Embodiment 22. The leriglitazone, or a pharmaceutically acceptable salt thereof, of Embodiment 21, wherein BW_{coef} is 0.75.

Embodiment 23. The leriglitazone, or a pharmaceutically acceptable salt thereof, of Embodiments 21 or 22, wherein AGE_{coef} is 0.131.

Embodiment 24. The leriglitazone, or a pharmaceutically acceptable salt thereof, of any one of Embodiments 21-23, wherein SEX_{coef} is 0.147.

Embodiment 25. The leriglitazone, or a pharmaceutically acceptable salt thereof, of any one of Embodiments 21-24, wherein BMI_{coef} is 0.541.

Embodiment 26. The leriglitazone, or a pharmaceutically acceptable salt thereof, of any one of Embodiments 21-25, wherein FOOD_{coef}=0.0477.

Embodiment 27. The leriglitazone, or a pharmaceutically acceptable salt thereof, of any one of Embodiments 21-26, wherein the disease or disorder is a CNS disease or disorder.

Embodiment 28. The leriglitazone, or a pharmaceutically acceptable salt thereof of any one of Embodiments 21-26, wherein the disease or disorder is a lung disease or disorder.

Embodiment 29. The leriglitazone, or a pharmaceutically acceptable salt thereof, of any one of Embodiments 21-28, wherein the target AUC_{target} is 100 to 135 μg·h/mL.

Embodiment 30. The leriglitazone, or a pharmaceutically acceptable salt thereof, of any one of Embodiments 1-8, wherein the target AUC_{target} is 150±20% μg·h/mL.

Embodiment 31. The leriglitazone, or a pharmaceutically acceptable salt thereof, of any one of Embodiments 21-28, wherein the target AUC_{target} is 170±20% μg·h/mL.

Embodiment 32. The leriglitazone, or a pharmaceutically acceptable salt thereof, of any one of Embodiments 21-28, wherein the target AUC_{target} is 190±20% μg·h/mL.

Embodiment 33. The leriglitazone, or a pharmaceutically acceptable salt thereof, of any one of Embodiments 21-28, wherein the target AUC_{target} is 200±20% μg·h/mL.

Embodiment 34. The leriglitazone, or a pharmaceutically acceptable salt thereof, of any one of Embodiments 21-28, wherein the target AUC_{target} is 220±20% μg·h/mL.

Embodiment 35. The leriglitazone, or a pharmaceutically acceptable salt thereof, of any one of Embodiments 21-26, wherein the disease or disorder is a liver disease or disorder.

Embodiment 36. The leriglitazone, or a pharmaceutically acceptable salt thereof, of Embodiment 35, wherein the AUC_{target} is 50±20% μg·h/mL.

Embodiment 37. The leriglitazone, or a pharmaceutically acceptable salt thereof, of any one of Embodiments 21-31, wherein the patient is a pediatric patient.

Embodiment 38. The leriglitazone, or a pharmaceutically acceptable salt thereof, of any one of Embodiments 1-36, wherein the patient is an adult patient.

Embodiment 39. The leriglitazone, or a pharmaceutically acceptable salt thereof, of any one of Embodiments 21-38, wherein SF is 0.7, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.9.

Embodiment 40. The method of any one of Embodiments 1-18, wherein SF is 0.7, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, or 0.9.

Embodiment 41. A method of treating a disease or disorder in a patient in need thereof, the method comprising administering 5-[[4-[2-[5-(1-hydroxyethyl)pyridin-2-yl]ethoxy]phenyl]methyl]-1,3-thiazolidine-2,4-dione (leriglitazone) or 5-[[4-[2-[5-(1-hydroxyethyl)pyridin-2-yl]ethoxy]phenyl]methyl]-1,3-thiazolidine-2,4-dione hydrochloride (leriglitazone HCl) to the patient, wherein the initial dose of leriglitazone or leriglitazone HCl, in mg, is determined according to Formula I:

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is 0.74 when leriglitazone is administered and 0.81 when leriglitazone HCl is administered;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 265 μg·h/mL of leriglitazone.

Embodiment 42. The method of Embodiment 41, wherein BW_{coef} is 0.75.

Embodiment 43. The method of Embodiments 41 or 42, wherein AGE_{coef} is 0.131.

Embodiment 44. The method of any one of Embodiments 41-43, wherein SEX_{coef} is 0.147.

Embodiment 45. The method of any one of Embodiments 41-44, wherein BMI_{coef} is 0.541.

Embodiment 46. The method of any one of Embodiments 41-45, wherein FOOD_{coef}=0.0477.

Embodiment 47. The method of any one of Embodiments 41-46, wherein the disease or disorder is a CNS disease or disorder.

Embodiment 48. The method of any one of Embodiments 41-46, wherein the disease or disorder is a lung disease or disorder.

Embodiment 49. The method of any one of Embodiments 41-48, wherein the target AUC_{target} is 100 to 135 μg·h/mL.

Embodiment 50. The method of any one of Embodiments 41-48, wherein the target AUC_{target} is 150±20% μg·h/mL.

Embodiment 51. The method of any one of Embodiments 41-48, wherein the target AUC_{target} is 170±20% μg·h/mL.

Embodiment 52. The method of any one of Embodiments 41-48, wherein the target AUC_{target} is 190±20% μg·h/mL.

Embodiment 53. The method of any one of Embodiments 41-48, wherein the target AUC_{target} is 200±20% μg·h/mL.

Embodiment 54. The method of any one of Embodiments 41-48, wherein the target AUC_{target} is 220±20% μg·h/mL.

Embodiment 55. The method of any one of Embodiments 41-46, wherein the disease or disorder is a liver disease or disorder.

Embodiment 56. The method of Embodiment 55, wherein the AUC_{target} is 50±20% μg·h/mL.

Embodiment 57. The method of any one of Embodiments 41-51, wherein the patient is a pediatric patient.

Embodiment 58. The method of any one of Embodiments 41-56, wherein the patient is an adult patient.

Embodiment 59. The method of any one of Embodiments 41-58, wherein leriglitazone is administered.

Embodiment 60. The method of Embodiments 41-59, wherein leriglitazone HCl is administered.

Embodiment 61. Leriglitazone or leriglitazone HCl for use in treating a disease or disorder in a patient in need thereof, wherein the initial dose of leriglitazone or leriglitazone HCl, in mg, to be administered is determined according to Formula I:

$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$

wherein:
SF is the salt factor, wherein SF is 0.74 when leriglitazone is administered and 0.81 when leriglitazone HCl is administered;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 265 μg·h/mL of leriglitazone.

Embodiment 62. The leriglitazone or leriglitazone HCl of Embodiment 61, wherein BW_{coef} is 0.75.

Embodiment 63. The leriglitazone or leriglitazone HCl of Embodiments 61 or 62, wherein AGE_{coef} is 0.131.

Embodiment 64. The leriglitazone or leriglitazone HCl of any one of Embodiments 61-63, wherein SEX_{coef} is 0.147.

Embodiment 65. The leriglitazone or leriglitazone HCl of any one of Embodiments 61-64, wherein BMI_{coef} is 0.541.

Embodiment 66. The leriglitazone or leriglitazone HCl of any one of Embodiments 61-65, wherein FOOD_{coef}=0.0477.

Embodiment 67. The leriglitazone or leriglitazone HCl of any one of Embodiments 61-66, wherein the disease or disorder is a CNS disease or disorder.

Embodiment 68. The leriglitazone or leriglitazone HCl of any one of Embodiments 61-66, wherein the disease or disorder is a lung disease or disorder.

Embodiment 69. The leriglitazone or leriglitazone HCl of any one of Embodiments 61-68, wherein the target AUC_{target} is 100 to 135 μg·h/mL.

Embodiment 70. The leriglitazone or leriglitazone HCl of any one of Embodiments 61-68, wherein the target AUC_{target} is 150±20% μg·h/mL.

Embodiment 71. The leriglitazone or leriglitazone HCl of any one of Embodiments 61-68, wherein the target AUC_{target} is 170±20% μg·h/mL.

Embodiment 72. The leriglitazone or leriglitazone HCl of any one of Embodiments 61-68, wherein the target AUC_{target} is 190±20% μg·h/mL.

Embodiment 73. The leriglitazone or leriglitazone HCl of any one of Embodiments 61-68, wherein the target AUC_{target} is 200±20% μg·h/mL.

Embodiment 74. The leriglitazone or leriglitazone HCl of any one of Embodiments 61-68, wherein the target AUC_{target} is 220±20% μg·h/mL.

Embodiment 75. The leriglitazone or leriglitazone HCl of any one of Embodiments 61-66, wherein the disease or disorder is a liver disease or disorder.

Embodiment 76. The leriglitazone or leriglitazone HCl of Embodiment 75, wherein the AUC_{target} is 50±20% μg·h/mL.

Embodiment 77. The leriglitazone or leriglitazone HCl of any one of Embodiments 21-31, wherein the patient is a pediatric patient.

Embodiment 78. The leriglitazone or leriglitazone HCl of any one of Embodiments 1-36, wherein the patient is an adult patient.

Embodiment 79. The leriglitazone or leriglitazone HCl of any one of Embodiments 21-38, wherein leriglitazone is to be administered.

Embodiment 80. The leriglitazone or leriglitazone HCl of Embodiments 21-38, wherein leriglitazone HCl is to be administered.

### VIII. References

1. Rodriguez-Pascau, L. et al., Sci. Transl. Med. 13, eabc0555 (2021).
2. Johnson, T. N. & Ke, A. B., J. Clin. Pharmacol. 61 Suppl 1, S83-S93 (2021).
3. Leong, R. et al., Clin. Pharmacol. Ther. 91, 926-931 (2012).
4. Zhao, P. et al., Clin. Pharmacol. Ther. 89, 259-267 (2011).
5. Upreti, V. V. & Wahlstrom, J. L., J Clin. Pharmacol. 56, 266-283 (2016).
6. Howgate, E. M., et al., Xenobiotica Fate Foreign Compd. Biol. Syst. 36, 473-497 (2006).
7. Inoue, S. et al., Xenobiotica Fate Foreign Compd. Biol. Syst. 36, 499-513 (2006).
8. Kusminski, C. M. et al., Diabetologia 50, 634-642 (2007).
9. Miyazaki, Y. et al., J. Clin. Endocrinol. Metab. 89, 4312-4319 (2004).
10. Qiu, D. & Li, X.-N., Int. J. Clin. Pharmacol. Ther. 53, 746-752 (2015).
11. Dupuis, L. et al., PloS One 7, e37885 (2012).
12. Kernan, W. N. et al., N. Engl. J. Med. 374, 1321-1331 (2016).
13. Viscoli, C. M. et al., J. Clin. Endocrinol. Metab. 102, 914-922 (2016).
14. Eckland, D. A. & Danhof, M., Exp. Clin. Endocrinol. Amp Diabetes 108, 234-242 (2000).
15. Boris, M. et al., J. Neuroinflammation 4, 3 (2007).
16. Capano, L. et al., Mol. Autism 9, 59 (2018).
17. Ibinez, L. et al., J. Adolesc. Health Off. Publ. Soc. Adolesc. Med. 61, 446-453 (2017).
18. Stabile, G. et al., J. Pediatr. Adolesc. Gynecol. 27, 177-182 (2014).
19. Tafuri, K. S. et al., J. Clin. Res. Pediatr. Endocrinol. 5, 236-239 (2013).
20. Christensen, M. L. et al., J. Clin. Pharmacol. 45, 1137-1144 (2005).
21. Johnson, T. N. et al., Drug Metab. Dispos. Biol. Fate Chem. 44, 1090-1098 (2016).
22. Johnson, T. N. & Rostami-Hodjegan, A., Paediatr. Anaesth. 21, 291-301 (2011).
23. Beck, F. et al., Proc. Biol. Sci. 247, 83-87 (1992)

Having now fully described this disclosure, it will be understood by those of ordinary skill in the art that the same can be performed within a wide and equivalent range of conditions, formulations, and other parameters without affecting the scope of the invention or any embodiment thereof.

Other embodiments of the disclosure will be apparent to those skilled in the art from consideration of the specification and practice of the invention disclosed herein. It is intended that the specification and examples be considered exemplary only, with a true scope and spirit of the invention being indicated by the following claims.

All patents, patent applications, and publications cited herein are fully incorporated by reference herein in their entirety.

What is claimed is:

1. 5-[[4-[2-[5-(1-Hydroxyethyl)pyridin-2-yl]ethoxy]phenyl]methyl]-1,3-thiazolidine-2,4-dione (leriglitazone), or a pharmaceutically acceptable salt thereof, preferably the HCl salt, for use in treating a disease or disorder in a patient in need thereof, wherein the initial dose of leriglitazone, or a pharmaceutically acceptable salt thereof, in mg, is determined according to Formula:
$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$
wherein:
SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 240 μg·h/mL of leriglitazone.

2. The leriglitazone, or a pharmaceutically acceptable salt thereof, of claim 1, wherein BW_{coef} is 0.75.

3. The leriglitazone, or a pharmaceutically acceptable salt thereof, of claim 1 or 2, wherein AGE_{coef} is 0.131.

4. The leriglitazone, or a pharmaceutically acceptable salt thereof, of any one of claims 1-3, wherein SEX_{coef} is 0.147.

5. The leriglitazone, or a pharmaceutically acceptable salt thereof, of any one of claims 1-4, wherein BMI_{coef} is 0.541.

6. The leriglitazone, or a pharmaceutically acceptable salt thereof, of any one of claims 1-5, wherein FOOD_{coef}=0.0477.

7. The leriglitazone, or a pharmaceutically acceptable salt thereof, of any one of claims 1-6, wherein the disease or disorder is a CNS disease or disorder, or a lung disease or disorder and the AUC_{target} is 170±20% μg·h/mL.

8. The leriglitazone, or a pharmaceutically acceptable salt thereof, of claim 7, wherein the patient is pediatric patient.

9. The leriglitazone, or a pharmaceutically acceptable salt thereof, of any one of claims 1-6, wherein the disease or disorder is a CNS disease or disorder, or a lung disease or disorder, the patient is an adult patient, and the AUC_{target} is 200±20% μg·h/mL.

10. The leriglitazone, or a pharmaceutically acceptable salt thereof, of any one of claims 1-6, wherein the disease or disorder is a liver disease or disorder and the AUC_{target} is 50±20% μg·h/mL.

11. The leriglitazone, or a pharmaceutically acceptable salt thereof, of any one of claims 1-10, wherein the amount of leriglitazone, or a pharmaceutically acceptable salt thereof, in mg, to be administered to the patient as the initial dose is to be administered to the patient in subsequent doses for up to six weeks.

12. Leriglitazone, or a pharmaceutically acceptable salt thereof, for use in treating a disease or disorder in a patient in need thereof, wherein the AUC of leriglitazone in the patient after a dose of leriglitazone, or a pharmaceutically acceptable salt thereof, is determined; and
(i) a higher dose of leriglitazone, or a pharmaceutically acceptable salt thereof, is to be administered to the patient if the AUC is less than 149 μg·h/mL;
(ii) a lower dose of leriglitazone, or a pharmaceutically acceptable salt thereof, is to be administered to the patient if the AUC is more than 191 μg·h/mL; and
(iii) the same dose of leriglitazone, or a pharmaceutically acceptable salt thereof, is to be administered to the patient if the AUC is between 150 and 190 μg·h/mL, wherein the patient is a pediatric patient.

13. Leriglitazone, or a pharmaceutically acceptable salt thereof, for use in treating a disease or disorder in a patient in need thereof, wherein:
(i) a 10 mL of an oral suspension comprising about 15 mg of leriglitazone, or a pharmaceutically acceptable salt thereof, preferably leriglitazone HCl, per mL is to be administered to the patient for 1-10 weeks; and
(ii) a 12 mL of an oral suspension comprising about 15 mg of leriglitazone, or a pharmaceutically acceptable salt thereof, preferably leriglitazone HCl, per mL is to be administered to the patient after (i).

14. The leriglitazone, or a pharmaceutically acceptable salt thereof, of any one of claims 1-6 or 11-13, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

15. The leriglitazone, or a pharmaceutically acceptable salt thereof, of claim 14, wherein the disease or disorder is a central nervous system disease or disorder.

16. The leriglitazone, or a pharmaceutically acceptable salt thereof, of any one of claims 7, 8, 9, or 15, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

17. The leriglitazone, or a pharmaceutically acceptable salt thereof, of claim 16, wherein the central nervous system disease or disorder is a neurodegenerative disease.

18. The leriglitazone, or a pharmaceutically acceptable salt thereof, of claim 17, wherein the neurodegenerative disease is selected from the group consisting of Alzheimer's disease, Huntington's chorea, Parkinson's disease, multiple sclerosis, neuromyelitis optica, leukodystrophy, amyotrophic lateral sclerosis (ALS), degenerative ataxia, multiple system atrophy, neurodegeneration and brain iron accumulation disorders (NBIA), neuromyopathy, and a motor neuron disease.

19. The leriglitazone, or a pharmaceutically acceptable salt thereof, of claim 18, wherein the leukodystrophy is X-linked adrenoleukodystrophy, adrenomyeloneuropathy, cerebral adrenoleukodystrophy, or metachromatic leukodystrophy.

20. The leriglitazone, or a pharmaceutically acceptable salt thereof, of claim 19, wherein the leukodystrophy is cerebral adrenoleukodystrophy.

21. The leriglitazone, or a pharmaceutically acceptable salt thereof, of claim 18, wherein the degenerative ataxia is Friedreich's ataxia.

22. The leriglitazone, or a pharmaceutically acceptable salt thereof, of claim 18, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

23. The leriglitazone, or a pharmaceutically acceptable salt thereof, of claim 16, wherein the central nervous system disorder is a cerebrovascular disease selected from the group consisting of global or local ischemia, intracerebral haemorrhage, stroke, and vascular dementia.

24. The leriglitazone, or a pharmaceutically acceptable salt thereof, of claim 16, the central nervous system disorder is a viral disease selected from the group consisting of meningitis, encephalitis, rabies, measles, mumps, poliomyelitis, herpes simplex, and varicella zoster.

25. The leriglitazone, or a pharmaceutically acceptable salt thereof, of claim 16, wherein the central nervous system disease or disorder is a rare metabolic disease selected from the group consisting of organic acidemias, fatty acid disorders and genetic mitochondrial disorders.

26. The leriglitazone, or a pharmaceutically acceptable salt thereof, of claim 14, wherein the disease or disorder is a mitochondrial disease.

27. The leriglitazone, or a pharmaceutically acceptable salt thereof, of claim 26, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX deficient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

28. The leriglitazone, or a pharmaceutically acceptable salt thereof, of claim 27, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

29. The leriglitazone, or a pharmaceutically acceptable salt thereof, of claim 27, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

30. The leriglitazone, or a pharmaceutically acceptable salt thereof, of any one of claims 7, 8, 9, or 14, wherein the lung disease or disorder is an inflammatory lung condition or disease caused by a viral infection, acute inflammation of the lung, or interstitial lung disease.

31. The leriglitazone, or a pharmaceutically acceptable salt thereof, of claim 30 for treating an inflammatory lung condition or disease caused by a viral infection in a patient in need thereof.

32. The leriglitazone, or a pharmaceutically acceptable salt thereof, of claim 31, wherein the viral infection is a human coronavirus infection, an influenza virus infection, or a HIV virus infection.

33. The leriglitazone, or a pharmaceutically acceptable salt thereof, of claim 32, wherein the human coronavirus is HCoV 229E, HCoV OC43, HCoV NL63, HCoV HKU1, SARS CoV, MERS CoV, or SARS CoV-2, or a mutated strain thereof.

34. The leriglitazone, or a pharmaceutically acceptable salt thereof, of claim 33, wherein the human coronavirus is SARS CoV-2.

35. The leriglitazone, or a pharmaceutically acceptable salt thereof, of claim 34, wherein the human coronavirus is a mutated strain of SARS CoV 2.

36. The leriglitazone, or a pharmaceutically acceptable salt thereof, of any one of claims 31-35, wherein the inflammatory lung condition or disease caused by the viral infection is hypercytokinaemia, haemophagocytic lymphohistiocytosis, pneumonia, acute respiratory distress syndrome, or systemic inflammatory response syndrome.

37. The leriglitazone, or a pharmaceutically acceptable salt thereof, of claim 36, wherein the inflammatory lung condition or disease caused by the viral infection is acute respiratory distress syndrome.

38. The leriglitazone, or a pharmaceutically acceptable salt thereof, of claim 30 for treating acute inflammation of the lung in a patient in need thereof.

39. The leriglitazone, or a pharmaceutically acceptable salt thereof, of claim 38, wherein the acute inflammation of the lung is caused by a bacterial infection.

40. The leriglitazone, or a pharmaceutically acceptable salt thereof, of claim 38, wherein the acute inflammation of the lung is pneumonia or acute respiratory distress syndrome.

41. The leriglitazone, or a pharmaceutically acceptable salt thereof, of claim 30 for treating interstitial lung disease in a patient in need thereof.

42. The leriglitazone, or a pharmaceutically acceptable salt thereof, of claim 41, wherein the interstitial lung disease is idiopathic pulmonary fibrosis.

43. The leriglitazone, or a pharmaceutically acceptable salt thereof, of claim 10 or 14, wherein the liver disease or disorder is nonalcoholic steatohepatitis (NASH) or nonalcoholic fatty liver disease (NAFLD).

44. A method of treating a disease or disorder in a patient in need thereof, the method comprising administering 5-[[4-[2-[5-(1-hydroxyethyl)pyridin-2-yl]ethoxy]phenyl]methyl]-1,3-thiazolidine-2,4-dione (leriglitazone), or a pharmaceutically acceptable salt thereof, to the patient, wherein the initial dose of leriglitazone, or a pharmaceutically acceptable salt thereof, in mg, is determined according to Formula I:
$$Initial Dose = {AUC}_{target} * S F * {( B W / 75 )}^{⋀} B {W}_{coef} * {( AGE / 38 )}^{⋀} {AGE}_{coef} * ( 1 - {SEX}_{coef} * SEX ) / ( {( B M I / 23.85 )}^{⋀} B M {I}_{coef} * ( 1 - {FOOD}_{coef} * FOOD ) ) , \qquad Formula I$$
wherein:
SF is from 0.7 to 0.9;
BW is the weight of the patient in kg;
AGE is the age of the patient in years;
SEX is the sex of the patient, wherein male is 1 and female is 2;
BMI is the body mass index in kg/m², derived from the BW and height of the patient;
FOOD is the food status of the patient, wherein fasted is 0 and fed is 1;
BW_{coef} is about 0.6 to about 0.9;
AGE_{coef} is about 0.02 to about 0.2;
SEX_{coef} is about 0.04 to about 0.25;
BMI_{coef} is about 0.3 to about 0.8;
FOOD_{coef} is about 0.02 to about 0.7; and
AUC_{target} is about 40 to about 240 μg·h/mL of leriglitazone.

45. The method of claim 44, wherein BW_{coef} is 0.75.

46. The method of claim 44 or 45, wherein AGE_{coef} is 0.131.

47. The method of any one of claims 44-46, wherein SEX_{coef} is 0.147.

48. The method of any one of claims 44-47, wherein BMI_{coef} is 0.541.

49. The method of any one of claims 44-48, wherein FOOD_{coef}=0.0477.

50. The method of any one of claims 44-49, wherein the disease or disorder is a CNS disease or disorder, or a lung disease or disorder and the AUC_{target} is 170±20% μg·h/mL.

51. The method of claim 50, wherein the patient is pediatric patient.

52. The method of any one of claims 44-49, wherein the disease or disorder is a CNS disease or disorder, or a lung disease or disorder, the patient is an adult patient, and the AUC_{target} is 200±20% μg·h/mL.

53. The method of any one of claims 44-49, wherein the disease or disorder is a liver disease or disorder and the AUC_{target} is 50±20% μg·h/mL.

54. The method of any one of claims 44-53, wherein the amount of leriglitazone, or a pharmaceutically acceptable salt thereof, in mg, administered to the patient as the initial dose is administered to the patient in subsequent doses for up to six weeks.

55. A method of treating a disease or disorder in a patient in need thereof, the method comprising determining the AUC of leriglitazone in the patient after a dose of leriglitazone, or a pharmaceutically acceptable salt thereof; and
(i) administering a higher dose of leriglitazone, or a pharmaceutically acceptable salt thereof, if the AUC is less than 149 μg·h/mL;
(ii) administering a lower dose of leriglitazone, or a pharmaceutically acceptable salt thereof, if the AUC is more than 191 μg·h/mL; and
(iii) administering the same dose of leriglitazone, or a pharmaceutically acceptable salt thereof, if the AUC is between 150 and 190 μg·h/mL, wherein the patient is a pediatric patient.

56. A method of treating a disease or disorder in a patient in need thereof, the method comprising:
(i) administering 10 mL of an oral suspension comprising about 15 mg of leriglitazone, or a pharmaceutically acceptable salt thereof, per mL to the patient for 1-10 weeks; and
(ii) administering 12 mL of an oral suspension comprising 15 mg of leriglitazone, or a pharmaceutically acceptable salt thereof, per mL to the patient after (i).

57. The method of any one of claims 44-49 or 53-56, wherein the disease or disorder is a central nervous system disease or disorder, mitochondrial disease, a liver disease or disorder, chronic granulomatous disorder, a polycystic ovary syndrome, a thyroid carcinoma, a thyroid autoimmune disorder, a pituitary adenoma, atherosclerosis, hypertension, a skin disease, an inflammation and autoimmune disease, an inflammatory respiratory disease, or a lung disease or disorder.

58. The method of claim 57, wherein the disease or disorder is a central nervous system disease or disorder.

59. The method of any one of claims 50, 51, 52, or 58, wherein the central nervous system disease or disorder is selected from the group consisting of neurodegenerative disease, cerebrovascular disease, seizure, epilepsy, viral disease, neuroinflammatory disease, brain tumor, organic acidemias, fatty acid disorder, and genetic mitochondrial disorder.

60. The method of claim 59, wherein the central nervous system disease or disorder is a neurodegenerative disease.

61. The method of claim 60, wherein the neurodegenerative disease is selected from the group consisting of Alzheimer's disease, Huntington's chorea, Parkinson's disease, multiple sclerosis, neuromyelitis optica, leukodystrophy, ALS, degenerative ataxia, multiple system atrophy, NBIA (neurodegeneration and brain iron accumulation disorders), neuromyopathy, and a motor neuron disease.

62. The method of claim 61, wherein the leukodystrophy is X-linked adrenoleukodystrophy, adrenomyeloneuropathy, cerebral adrenoleukodystrophy, or metachromatic leukodystrophy.

63. The method of claim 62, wherein the leukodystrophy is cerebral adrenoleukodystrophy.

64. The method of claim 61, wherein the degenerative ataxia is Friedreich's ataxia.

65. The method of claim 61, wherein the motor neuron disease is selected from the group consisting of progressive bulbar palsy, pseudobulbar palsy, primary lateral sclerosis (PLS), progressive muscular atrophy, spinal muscular atrophy (SMA), post-polio syndrome (PPS)-Marie-Tooth disease, Guillan-Barré syndrome, and adrenomyeloneuropathy (AMN).

66. The method of claim 59, wherein the central nervous system disorder is a cerebrovascular disease selected from the group consisting of global or local ischemia, intracerebral haemorrhage, stroke, and vascular dementia.

67. The method of claim 59, the central nervous system disorder is a viral disease selected from the group consisting of meningitis, encephalitis, rabies, measles, mumps, poliomyelitis, herpes simplex, and varicella zoster.

68. The method of claim 59, wherein the central nervous system disease or disorder is a rare metabolic disease selected from the group consisting of organic acidemias, fatty acid disorders and genetic mitochondrial disorders.

69. The method of claim 57, wherein the disease or disorder is a mitochondrial disease.

70. The method of claim 69, wherein the mitochondrial disease is a primary mitochondrial disorder selected from the group consisting of Rett syndrome, Alper's disease; Leber's hereditary optic neuropathy (LHON); Kearns-Sayre syndrome (KSS); Leigh's syndrome; Leigh-like syndrome; maternally inherited Leigh syndrome (MILS); mitochondrial depletion syndrome (MDS); mitochondrial DNA depletion syndrome (MDDS); mitochondrial encephalomyopathy; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; chronic progressive external opthalmoplegia (CPEO); dominant optic atrophy (DOA); autosomal dominant optic atrophy (ADOA); mitochondrial myopathy; cardiomyopathy; mitochondrial encephalopathy; myoclonic epilepsy; maternally inherited diabetes and deafness (MIDD); ataxia neuropathy spectrum; 3-methylglutaconic aciduria; sensoneural deafness; neuroradiological findings of Leigh-like syndrome (MEGDEL); SURF1 (COX deficient Leigh syndrome due to complex IV surfeit protein deficiency); oxidative phosphorylation disorders; Berth syndrome; lethal infantile cardiomyopathy (LIC); pyruvate carboxylase deficiency; pyruvate dehydrogenase deficiency; POLG mutation; isolated or combined OXPHOS deficiencies with so far unsolved genetic defect including disturbed pyruvate oxidation and ATP plus PCr production rates; POLG2 mutation; carnitine-acyl-cartinine deficiency; carnitine deficiency; creatinine deficiency syndromes; Co-Enzyme Q10 deficiency; Complex I deficiency; Complex II deficiency; Complex III deficiency; Complex IV deficiency; Complex V deficiency; lactic acidosis; leukoencephalopathy with brain stem and spinal cord involvement and lactate elevation (LBSL); Luft disease; carnitine palmitoyltransferase (CPT I or CPT II) deficiency; short-chain acyl-CoA dehydrogenase deficiency (SCAD); short-chain 3-hydroxyacetyl-CoA dehydrogenase deficiency (SCHAD); medium-chain acyl-CoA dehydrogenase deficiency (MCAD); multiple acyl-CoA dehydrogenase deficiency (MADD); long-chain acyl-CoA dehydrogenase deficiency (LCAD); very long-chain acyl-CoA dehydrogenase deficiency (VLCAD); trifunctional protein (TFP) deficiency; and glutaric aciduria Type II.

71. The method of claim 70, wherein the mitochondrial disease is selected from the group consisting of Rett syndrome; dominant optic atrophy (DOA); autosomal dominant optic atrophy (ADOA); Complex I deficiency; Leber hereditary optic neuropathy (LHON); Kearns-Sayre syndrome (KSS); Leigh's syndrome; mitochondrial encephalomyopathy with lactic acidosis and stroke-like episodes (MELAS); myoclonic epilepsy with ragged red fibers (MERRF); mitochondrial neurogastrointestinal encephalopathy syndrome (MNGIE); neuropathy, ataxia, and retinitis pigmentosa (NARP); Pearson syndrome; and chronic progressive external opthalmoplegia (CPEO).

72. The method of claim 70, wherein the mitochondrial disease is a secondary mitochondrial disorder selected from the group consisting of Duchenne muscular dystrophy (DMD); Becker muscular dystrophy (BMD); myotonic dystrophy (BMD); congenital myopathies; glycogen storage disorders; spinal-bulbar muscular atrophy (SBMA); argininosuccinic aciduria; autism spectrum disorder (ASD); autoimmune diseases of the skin (such as pemphigus vulgaris and lupus); methylmalonic and propionic acidurias; disorders or purine and/or pyrimidine synthesis; facioscapulohumeral muscular dystrophy (FSHD); congenital muscular dystrophies; collagen VI muscular dystrophies (e.g., Ullrich congenital muscular dystrophy, Bethlem myopathy, oculopharyngeal distal and Emery-Dreifuss); DiGeorge syndrome; and neuromuscular disorders (such as limb-girdle muscular dystrophy, inflammatory myopathies, Charcot Marie Tooth (CMT) neuropathy, and drug-induced peripheral neuropathies).

73. The method of any one of claims 50, 51, 52, or 57, wherein the lung disease or disorder is an inflammatory lung condition or disease caused by a viral infection, acute inflammation of the lung, or interstitial lung disease.

74. The method of claim 73 for treating an inflammatory lung condition or disease caused by a viral infection in a patient in need thereof.

75. The method of claim 74, wherein the viral infection is a human coronavirus infection, an influenza virus infection, or a HIV virus infection.

76. The method of claim 75, wherein the human coronavirus is HCoV 229E, HCoV OC43, HCoV NL63, HCoV HKU1, SARS CoV, MERS CoV, or SARS CoV-2, or a mutated strain thereof.

77. The method of claim 76, wherein the human coronavirus is SARS CoV-2.

78. The method of claim 77, wherein the human coronavirus is a mutated strain of SARS CoV 2.

79. The method of any one of claims 74-78, wherein the inflammatory lung condition or disease caused by the viral infection is hypercytokinaemia, haemophagocytic lymphohistiocytosis, pneumonia, acute respiratory distress syndrome, or systemic inflammatory response syndrome.

80. The method of claim 79, wherein the inflammatory lung condition or disease caused by the viral infection is acute respiratory distress syndrome.

81. The method of claim 73 for treating acute inflammation of the lung in a patient in need thereof.

82. The method of claim 81, wherein the acute inflammation of the lung is caused by a bacterial infection.

83. The method of claim 83, wherein the acute inflammation of the lung is pneumonia or acute respiratory distress syndrome.

84. The method of claim 73 for treating interstitial lung disease in a patient in need thereof.

85. The method of claim 83, wherein the interstitial lung disease is idiopathic pulmonary fibrosis.

86. The method of claim 53 or 57, wherein the liver disease or disorder is nonalcoholic steatohepatitis (NASH) or nonalcoholic fatty liver disease (NAFLD).

87. The leriglitazone, or pharmaceutically acceptable salt thereof, of any one of claims 1-43, wherein the leriglitazone, or pharmaceutically acceptable salt thereof, is leriglitazone HCl.

88. The leriglitazone, or pharmaceutically acceptable salt thereof, of any one of claims 1-11 or 14-43, wherein the leriglitazone, or pharmaceutically acceptable salt thereof, is leriglitazone HCl and SF is 0.81.

89. The method of any one of claims 44-86, wherein leriglitazone HCl is administered to the patient.

90. The method of any one of claims 44-54, 57-86, or 89, wherein SF is 0.81.
